(19) United States
(12) Patent Application Publication
(10) Pub. No.: US 2009/0280200 A1
(43) Pub. Date: Nov. 12, 2009

(54) HERBAL EXTRACTS WHICH INDUCE IMMUNE CELLS TO PRODUCE INTERFERON AND ACTIVATE TOLL-LIKE RECEPTORS

(75) Inventors: I Horng PAN, Hsinchu City (TW); Lain-Tze LEE, Hsinchu City (TW); Chih-Lung CHEN, Taichung County (TW); Hsin-Jan YAO, Yunlin County (TW); Chu-Hsun LU, Kaohsiung City (TW); Chen Hsuan LIN, Pingtung County (TW); Ya-Yan YANG, Taichung County (TW); Hsin-Hsin SHEN, Hsinchu (TW); Ming-Feng CHEN, Changhua County (TW)

Correspondence Address: LOWE HAUPTMAN HAM & BERNER, LLP, 1700 DIAGONAL ROAD, SUITE 300, ALEXANDRIA, VA, 22314, US

(73) Assignee: INDUSTRIAL TECHNOLOGY RESEARCH INSTITUTE, Hsinchu (TW)

(21) Appl. No.: 12/164,958

(22) Filed: Jun. 30, 2008

(30) Foreign Application Priority Data
May 8, 2008 (TW) 97116973

(51) Int. Cl. A61K 36/48; A61K 36/00; A61P 37/04

(52) U.S. Cl. 424/757; 424/773

(57) ABSTRACT

An herbal extract which induces immune cells to produce interferon and activates Toll-like receptors and a preparation method thereof are provided. The herbal extract is extracted from an effective amount of raw material including Glycyrrhizae Radix, Bupleuri Radix, Scutellariae Radix, Schisandrae Fructus and Paeoniae Rubra Radix. Glycyrrhizae Radix, Bupleuri Radix, Scutellariae Radix, Schisandrae Fructus and Paeoniae Rubra Radix have a weight ratio of 1-5:1-5:1-5:1-3:1-3.

### CROSS REFERENCE TO RELATED APPLICATIONS

This Application claims priority of Taiwan Patent Application No. 97116973, filed on May 8, 2008, the entirety of which is incorporated by reference herein.

### BACKGROUND OF THE INVENTION

1. Field of the Invention

The invention relates to an herbal extract, and in particular relates to an herbal extract which induces immune cells to produce interferon and activates Toll-like receptors and a preparation method thereof.

2. Description of the Related Art

Interferon (IFN), the first cytokine discovered, which was capable of interfering with virus replication, was identified by British virologist Alick Isaacs and the Swiss researcher Jean Lindenmann in 1957. When cells are infected by viruses, interferon is immediately produced to fight against the virus infection and simultaneously warn neighboring normal cells to prevent virus invasion. Interferon is divided into two groups. Type I interferon comprises interferon-α, interferon-β, interferon-ω and interferon-τ and type II interferon comprises interferon-γ. Interferon-α and interferon-β are produced in most cells. Interferon-γ, however, is merely produced in a part of some immune cells, for example, natural killer cells, CD4⁺T helper 1 (T_{H}1) lymphocytes and CD8⁺ cytotoxic T lymphocytes.

During virus infection, type I interferon is rapidly produced and binds to type I interferon receptors to start a JAK-STAT signal transduction pathway to open a interferon-stimulated gene (ISG) expression. An interferon-stimulated protein is the best weapon of interferon, to fight against virus infection. Currently, more than 500 interferon-stimulated proteins have been identified, widely participating in, for example, anti-virus, apoptosis, protein degradation, inflammatory cell response and lipid metabolism. Common interferon-stimulated proteins comprise protein kinase R (PKR), adenosine deaminase acting on RNA (ADAR), 2′,5′-oligo adenylate synthetase (OAS), RNase L and Mx protein. PKR inhibits eukaryotic initiation factor 2 (eIF2) to block virus protein synthesis. ADAR inhibits RNA editing. OAS and RNase L collapse virus RNA. Mx protein inhibits virus replication.

In addition to fighting against virus infection, type I interferon participates in immunomodulatory treatment, taking an important role in innate immune response and adaptive immune response. Type I interferon induces immune cells to produce IL-15 to promote survival and proliferation of natural killer cells and simultaneously stimulates MHC, CD80, CD86 and CD40 molecules to mature dendritic cells. Additionally, type I interferon induces differentiation of plasmacytoid dendritic cells (pDC) to form mature antigen presenting cells. In adaptive immune response, type I interferon plays an important role in activity of CD8⁺ cytotoxic T lymphocytes, survival of CD4⁺ T helper 1 (T_{H}1) lymphocytes and CD8⁺ cytotoxic T lymphocytes, and differentiation and proliferation of B lymphocytes.

Type I interferon has been successfully utilized in clinical therapy, benefiting anti-virus, cell growth regulation and immunomodulatory treatments. Examples of beneficial clinical therapy treatments were seen for viral diseases such as chronic hepatitis B, chronic hepatitis C, condyloma and Kaposi's sarcoma, hematological diseases such as hairy cell leukemia, chronic myeloid leukemia, multiple myeloma and Non-Hodgkin's lymphoma, and other tumors such as melanoma, renal cell carcinoma and basal cell carcinoma.

In accordance with current research, interferon is produced by activation of Toll-like receptors (TLR). Toll was found from fruit fly in 1988. Toll-like receptors were subsequently found from mammals. When a foreign pathogen invades an entity, Toll-like receptors recognize pathogen-associated molecular patterns (PAMPs) thereof to open a downstream gene expression through a signal transduction pathway, for example, by formation of cytokine (type I interferon, IL-1, IL-12 and interferon-α), chemotactic substance, MHC and co-stimulatory molecules. Toll-like receptors induce inducible nitric oxide synthase (iNOS) and antimicrobial peptide expression to start an innate immune response to directly damage the foreign pathogen. Furthermore, Toll-like receptors induce maturation of antigen presenting cells (dendritic cells) to activate adaptive immune response. When a microorganism invades an entity, Toll-like receptors of dendritic cells recognize pathogen and present pathogen's antigen on a cell surface through an MHC molecule. Native T lymphocytes are then activated and differentiated to the Th₁ lymphocytes by stimulation of IL-12 produced by inducing dendritic cells by the Toll-like receptors to start the adaptive immune response against the chronic virus infection, providing powerful and specific immune protection.

Currently known Toll-like receptors comprise the Toll-like receptor 1 to Toll-like receptor 10, which are capable of recognizing various foreign substances, for example, bacteria, virus, fungus and protozoan. A Toll-like receptor structure has two parts comprising an extracellular leucine-rich repeat (LRR) and an intracellular Toll-interleukin-1 receptor (TIR) domain. The extracellular leucine-rich repeat (LRR) recognizes foreign substances. The intracellular Toll-interleukin-1 receptor (TIR) domain combines with downstream adaptor proteins, for example, myeloid differentiation factor-88 (MyD88), TIR-associated protein (TIRAP/MAL), Toll/IL-1 receptor domain-containing adaptor inducing IFN-β (TRIF/TICAM-1) and Toll-receptor-associated molecule (TRAM/TIRP/TICAM-2), to activate the extracellular signal-regulated kinase (ERK), p38, c-Jun N-terminal kinase and NF-kB to induce formation of pro-inflammatory cytokines such as IL-1, IL-6, interferon-α and type I interferon.

In mammals, the Toll-like receptor 4, which is expressed in many immune cells, for example, B lymphocytes, dendritic cells, monocytes, macrophages, granulocytes and T lymphocytes, is capable of recognizing various foreign viruses such as respiratory syncytial virus (RSV), hepatitis C virus (HCV) and mouse mammary tumor virus (MMTV), and induces immune cells to produce considerable quantities of interferon through combining with MyD88 and TIRAP.

Among overall Toll-like receptors, the Toll-like 2 recognizes maximum PAMPs, for example, lipoarabinomannan (LAM), lipopolysaccharide (LPS), lipoteichoic acid (LTA), peptidoglycan (PGN) and other lipoproteins, glycolipids and glycoproteins, mostly deriving from bacteria. Also, the Toll-like receptor 2 recognizes virus invasion, for example, the measles virus (MV), human cytomegalovirus (HCMV) and hepatitis C virus.

The Toll-like receptor 7 is highly expressed in monocytes, B lymphocytes and dendritic cells. Once foreign substance invasion is recognized, considerable quantities of type I interferon are produced, especially interferon-α, which plays an important role in innate immune response. The Toll-like receptor 7 detects G/U-rich ssRNA, which is derived from viruses, for example, human immunodeficiency virus (HIV) and VSV.

With the development of drugs, the Toll-like receptor 9 antagonist has entered clinical trials, which stimulates dendritic cells to produce IL-12 and considerable quantities of interferon-α and induces B lymphocyte proliferation and antibody secretion, with a good curative effect on HVC patients whom did not positively react to the conventional interferon-α treatment. Toll-like receptor 9 antagonist induces cells to exhibit a wide antiviral response and release various cytokines, especially interferon-α, achieving a high virus scavenging efficiency on various HVC genotype patients.

Currently, various antiviral drugs have been developed and widely applied in clinical therapy, for example, ribavirin, a nucloside analog, inhibiting growth of various viruses such as respiratory syncytial virus, influenza virus, adenovirs, HIV and HCV, amantadine, inhibiting M2 membrane proteins of influenza virus A to block virus replication, and zidovudine (AZT), didanosine (ddI), zalcitabine (ddc), stavudine (d4T) and lamivudine (3TC), inhibiting reverse transcriptase of HIV to block reverse transcription from RNA to DNA so as to break DNA synthesis.

Although many antiviral drugs have been effectively utilized in clinical therapy, the problem of drug resistance gradually occurs, due to virus gene mutation such that binding targets for antiviral drugs disappear. For example, mutation of the thymidine kinase gene of the HSV results in blocking of the conversion from acyclovir and ganciclovir to effective substances in cells, thus forming drug resistance to such drugs. Additionally, mutation of the M2 membrane protein gene of influenza virus A results in formation of drug resistance to amantadine and rimantadine, mutation of the reverse transcriptase or protease gene of HIV results in formation of drug resistance, and mutation of the non-structural 5A, envelope gene and 2-glycoprotein gene of HCV results in formation of drug resistance to interferon.

Currently, more and more drug research have focused on immunomodulatory treatments. A foreign microorganism is scavenged by stimulating an innate immune response and adaptive immune response of host cells. Herbs have an advantage in immunomodulatory treatment, because they have no deteriorating effect on normal organisms but an improved effect (over other conventional methods) on immune-disordered organisms, wherein an abnormal immune state can be corrected for recovery and immune stability is retained. Additionally, immunity of normal organisms may be simultaneously improved.

After thousands of years of consideration, herbs, possessing a natural structure and activity, have been proven to be a drug with determined curative effect.

In clinical trials, herbs exhibited prominent curative effects on viral diseases with various pathogenicity, latency periods and infection routes, for example, for viral diseases such as hepatitis B, hepatitis C, influenza, epidemic parotitis, epidemic cerebrospinal meningitis, viral hepatitis, enterovirus and AIDS.

U.S. Pat. No. 6,787,165 discloses a composition comprising a supercritical extract of Flos Lonicerae and Fructus Forsythiae, an aqueous extract of Flos Lonicerae and Fructus Forsythiae, and an aqueous extract of Scutellariae Radix, which inhibited the influenza virus. U.S. Pat. No. 6,696,094 discloses a pharmaceutical composition containing aqueous extracts of fourteen (14) herbal ingredients including Hedyotis Diffuse and so on, with the form of an intravenous injection solution or capsules, exhibiting an anti-HIV effect in clinical trials. U.S. Pat. No. 6,214,350 discloses an herbal prescription (HHT888-4) containing Flos Lonicerae, Glycyrrhizae Radix and Solanum Nigrum, which inhibited HIV activity to achieve over 98% in lymphocytes in vitro trials. U.S. Pat. No. 6,426,098 discloses an herbal extract comprising Curcuma Longa, Astragalus Membranaceus, Loranthus Parasiticus and Polygonum Cuspidatum, which possessed a prominent curative effect on hepatitis C in clinical trials.

Additionally, utilization of herbs have been incorporated in western medicine treatments. U.S. Pat. No. 2003/0211180 discloses an herbal composition (PHY906) comprising Ziziphus, which is utilized with chemotherapy drugs to increase curative effects on viral diseases, improve quality of life and reduce toxicity and negative side effects. The herbal composition has already entered the phase-II clinical trials in the U.S. Taiwan Pat. 1258373 discloses utilization of a hepatitis C assisted drug comprising Cordyceps Sinensisand Astragalus Membranaceus, which is incorporated with a hepatitis C composite treatment (interferon and ribavirin) to achieve over 70% virus scavenging efficiency. In addition to curative effect, recurrence is reduced.

Induction of interferon is significant for viral hepatitis treatments. WO 02102395 discloses an antiviral effect of an herbal extract comprising Nerium Indicum Mill and Glycyrrhizae Radix on HEp-2 cells infected by encephalo-myocarditis virus (EMCV). EMCV is effectively inhibited. In vitro, interferon-γ is also produced thereby.

There are ten Toll-like receptors (TLRs) in the human immune system. When suffering from the threat of pathogens (virus, bacteria or fungus), individual TLRs recognize pathogens and start corresponding immune responses. Binding between TLRs and pathogens are specific, inducing a series of reactions to protect cells from pathogen invasion. In TLR treatment, due to a high specificity of TLR to immune response, stimulators (agonists) or blockers (antagonists) are specifically administrated to induce self-immunomodulatory to achieve curative effect. Due to the high specificity of the treatment, in addition to continuous inhibition of pathogens, side effects and disorders resulting from non-specific activation of the innate immune system are reduced.

Actilon (a TLR drug), a synthetic short-chain oligonucleotides (ODNs) compound, has been developed by the Coley Pharmaceutical Group. The short-chain ODNs compound is similar to the DNA sequence, with a CpG structure of pathogen recognizable by TLR-9. After presentation of TLR-9 and a series of signal transduction pathway, interferon is produced to treat a viral disease, for example, hepatitis C. Actilon has entered phase-Ib clinical trials. Additionally, ANA245 and ANA975 (oral form) developed by Anadys Pharmaceutical Corporation is utilized to induce TLR-7 presentation. They have respectively entered phase-Ib and phase-Ia clinical trials.

Also, herbal extracts can be utilized in TLR treatments. WO 04093518 discloses a melanin extract of Echinacea, which was tested in monocyte (NF-kappa B/luciferase) in vitro. The melanin extract induced presentation of TLR-2 and TLR-4.

### BRIEF SUMMARY OF THE INVENTION

One embodiment of the invention provides an herbal extract which induces immune cells to produce interferon and activates Toll-like receptors, extracted from an effective amount of raw material comprising Glycyrrhizae Radix, Bupleuri Radix and Scutellariae Radix, wherein Glycyrrhizae Radix, Bupleuri Radix and Scutellariae Radix have a weight ratio of 1-5:1-5:1-5 or 1-2:1-2:1-2.

Glycyrrhizae Radix comprises Glycyrrhiza uralensis or Glycyrrhiza glabra. Bupleuri Radix comprises Bupleurum chinense or Bupleurum scorzonerifolium. Scutellariae Radix comprises Scutellaria baicalensis.

In an embodiment, the raw material of the herbal extract further comprises an effective amount of Schisandrae Fructus and Paeoniae Rubra Radix. Glycyrrhizae Radix, Bupleuri Radix, Scutellariae Radix, Schisandrae Fructus and Paeoniae Rubra Radix have a weight ratio of 1-5:1-5:1-5:1-3:1-3 or 1-2:1-2:1-2:1-2:1-2. Schisandrae Fructus comprises Schisandra chinensis or Schisandra sphenanthera. Paeoniae Rubra Radix comprises Paeonia lactiflora or Paeonia veitchii.

The herbal extract activates various Toll-like receptors, for example, Toll-like receptor 2, Toll-like receptor 4 and Toll-like receptor 7.

The herbal extract strengthens immune regulation, suitable for use in treatment of viral infectious diseases.

One embodiment of the invention provides a method for preparing an herbal extract which induces immune cells to produce interferon and activates Toll-like receptors, comprising providing an herbal composition comprising an effective amount of Glycyrrhizae Radix, Bupleuri Radix and Scutellariae Radix, wherein Glycyrrhizae Radix, Bupleuri Radix and Scutellariae Radix have a weight ratio of 1-5:1-5:1-5 or 1-2:1-2:1-2, extracting the herbal composition with a solvent to form an extract solution, concentrating the extract solution to form a concentrated product, drying the concentrated product and adding an excipient to prepare a specific formulation.

Glycyrrhizae Radix comprises Glycyrrhiza uralensis or Glycyrrhiza glabra. Bupleuri Radix comprises Bupleurum chinense or Bupleurum scorzonerifolium. Scutellariae Radix comprises Scutellaria baicalensis.

In an embodiment, the herbal composition further comprises an effective amount of Schisandrae Fructus and Paeoniae Rubra Radix. Glycyrrhizae Radix, Bupleuri Radix, Scutellariae Radix, Schisandrae Fructus and Paeoniae Rubra Radix have a weight ratio of 1-5:1-5:1-5:1-3:1-3 or 1-2:1-2:1-2:1-2:1-2. Schisandrae Fructus comprises Schisandra chinensis or Schisandra sphenanthera. Paeoniae Rubra Radix comprises Paeonia lactiflora or Paeonia veitchii.

The solvent comprises water or 0.1-95% ethanol. The solvent and the herbal composition have a weight ratio of 6:1-10:1. The concentrated product has a solid content of 10-30%. The concentrated product is dried by vacuum drying, freeze drying, spray drying or fluidized bed drying. The excipient comprises starch, maltose, lactose, sucrose, mannitol, magnesium stearate, silicon dioxide, microcrystalline cellulose, carboxymethyl cellulose or talc powder. The specific formulation comprises capsule, tablet, powder or fluid.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawing, wherein:

FIG. 1A shows cell toxicity of the herbal extract in neutrophil according to an embodiment of the invention.

FIG. 1B shows cell toxicity of the herbal extract in T lymphocytes according to an embodiment of the invention.

FIG. 2A shows quantity of interferon-γ produced by inducing natural killer cells and T lymphocytes by the herbal extract according to an embodiment of the invention.

FIG. 2B shows quantity of interferon-α produced by inducing natural killer cells by the herbal extract according to an embodiment of the invention.

FIG. 2C shows quantity of interferon-α produced by inducing T lymphocytes by the herbal extract according to an embodiment of the invention.

FIG. 3 shows quantity of interferon-γ induced by the herbal extract in a subject according to an embodiment of the invention.

FIGS. 4A-4I show expressions of Toll-like receptors of PBMC/T cells activated by the herbal extract according to an embodiment of the invention.

FIGS. 5A-5I show expressions of Toll-like receptors of PBMC/T cells activated by the herbal extract according to an embodiment of the invention.

FIGS. 6A-6I show expressions of Toll-like receptors of PBMC/T cells activated by the herbal extract according to an embodiment of the invention.

FIGS. 7A-7I show expressions of Toll-like receptors of PBMC/T cells activated by the herbal extract according to an embodiment of the invention.

FIGS. 8A-8I show expressions of Toll-like receptors of PBMC/T cells activated by the herbal extract according to an embodiment of the invention.

FIGS. 9A-9I show expressions of Toll-like receptors of PBMC/T cells activated by the herbal extract according to an embodiment of the invention.

FIGS. 10A-10I show expressions of Toll-like receptors of PBMC/T cells activated by the herbal extract according to an embodiment of the invention.

FIGS. 11A-11I show expressions of Toll-like receptors of PBMC/T cells activated by the herbal extract according to an embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides an herbal extract which induces immune cells to produce interferon and activates Toll-like receptors, extracted from an effective amount of raw material comprising Glycyrrhizae Radix, Bupleuri Radix and Scutellariae Radix.

Glycyrrhizae Radix, Bupleuri Radix and Scutellariae Radix have a weight ratio of about 1-5:1-5:1-5 or 1-2:1-2:1-2.

Glycyrrhizae Radix may comprise Glycyrrhiza uralensis or Glycyrrhiza glabra. Bupleuri Radix may comprise Bupleurum chinense or Bupleurum scorzonerifolium. Scutellariae Radix may comprise Scutellaria baicalensis.

In an embodiment, the raw material of the herbal extract may further comprise an effective amount of Schisandrae Fructus and Paeoniae Rubra Radix. Glycyrrhizae Radix, Bupleuri Radix, Scutellariae Radix, Schisandrae Fructus and Paeoniae Rubra Radix have a weight ratio of about 1-5:1-5:1-5:1-3:1-3 or 1-2:1-2:1-2:1-2:1-2. Schisandrae Fructus may comprise Schisandra chinensis or Schisandra sphenanthera. Paeoniae Rubra Radix may comprise Paeonia lactiflora or Paeonia veitchii.

The herbal extract activates various Toll-like receptors, for example, Toll-like receptor 2, Toll-like receptor 4 and Toll-like receptor 7.

One embodiment of the invention provides a method for preparing an herbal extract which induces immune cells to produce interferon and activates Toll-like receptors, comprising the following steps. An herbal composition comprising an effective amount of Glycyrrhizae Radix, Bupleuri Radix and Scutellariae Radix is provided. Glycyrrhizae Radix, Bupleuri Radix and Scutellariae Radix have a weight ratio of about 1-5:1-5:1-5 or 1-2:1-2:1-2. The herbal composition is then extracted with a solvent to form an extract solution. Next, the extract solution is concentrated to form a concentrated product. The concentrated product is then dried. Next, an excipient is added to prepare a specific formulation.

Glycyrrhizae Radix may comprise Glycyrrhiza uralensis or Glycyrrhiza glabra. Bupleuri Radix may comprise Bupleurum chinense or Bupleurum scorzonerifolium. Scutellariae Radix may comprise Scutellaria baicalensis.

In an embodiment, the herbal composition may further comprise an effective amount of Schisandrae Fructus and Paeoniae Rubra Radix. Glycyrrhizae Radix, Bupleuri Radix, Scutellariae Radix, Schisandrae Fructus and Paeoniae Rubra Radix have a weight ratio of about 1-5:1-5:1-5:1-3:1-3 or 1-2:1-2:1-2:1-2:1-2. Schisandrae Fructus may comprise Schisandra chinensis or Schisandra sphenanthera. Paeoniae Rubra Radix may comprise Paeonia lactiflora or Paeonia veitchii.

The solvent may comprise water or 0.1-95% ethanol. The solvent and the herbal composition have a weight ratio of about 6:1-10:1. The concentrated product has a solid content of about 10-30%. The concentrated product is dried by, for example, vacuum drying, freeze drying, spray drying or fluidized bed drying. The excipient may comprise starch, maltose, lactose, sucrose, mannitol, magnesium stearate, silicon dioxide, microcrystalline cellulose, carboxymethyl cellulose or talc powder. The specific formulation may comprise capsule, tablet, powder or fluid.

A raw material comprising Glycyrrhizae Radix, Bupleuri Radix, Scutellariae Radix, Schisandrae Fructus and Paeoniae Rubra Radix with a specific weight ratio is provided. The raw material is then extracted with 0.1-95% ethanol one or more times to form an extract solution. The weight of the ethanol is 6-fold to 10-fold times the raw material. Next, the extract solution is concentrated to form a concentrated product with a solid content of 10-30%. An excipient is then added and the product was freeze-dried to form a dried product with a water content of 2-10%. Next, the dried product is grounded to form powders with a diameter of less than 35 mesh. The dried herbal extract powders are then filled in a hard capsule. Each capsule is filled with about 1.35±0.09 g of the herbal extracts. The herbal extract is concentrated to 2-fold to 3-fold times the original content.

In the invention, interferon production and Toll-like receptor expression, for example, Toll-like receptor 2, Toll-like receptor 4 and Toll-like receptor 7 expressions, in neutrophil, T lymphocytes and natural killer cells are observed. The results act as a drug screening basis.

Herbal Extract Preparation

### EXAMPLE 1

A solution containing 4 kg Scutellaria baicalensis, 4 kg Bupleurum chinense, 4 kg Glycyrrhiza uralensis, 4 kg Paeonia lactiflora, 4 kg Schisandra chinensis (a weight ratio of 1:1:1:1:1) and 200 kg ethanol (30%) was thermal-extracted with reflux for one hour. After two extractions, an extract solution was formed and filtered through a 100mesh. After reduced pressure concentration, a concentrated product with a 15% solid content was formed. 2 kg dextrin-maltose was then added and the product was freeze-dried to form a dried product. Next, the dried product was grounded to form powders with a diameter of less than 35 mesh and then the product was blended with 120 g silicon dioxide and 60 g magnesium stearate. The dried herbal extract powders were then filled in a 0# hard capsule. The filling amount of each capsule was 565±40 mg, approximately 1.35±0.09 g of herbal extracts.

### EXAMPLE 2

A solution containing 2.5 g Scutellaria baicalensis, 2.5 g Bupleurum chinense, 2.5 g Glycyrrhiza uralensis, 7.5 g Paeonia lactiflora, 7.5 g Schisandra chinensis (a weight ratio of 1:1:1:3:3) and 250 g ethanol (30%) was thermal-extracted with reflux for one hour. After two extractions, an extract solution was formed and filtered through a 100mesh. After reduced pressure concentration and freeze-drying, a dried product was grounded to form powders.

### EXAMPLE 3

A solution containing 12.5 g Scutellaria baicalensis, 6.25 g Bupleurum chinense, 6.25 g Glycyrrhiza uralensis (a weight ratio of 2:1:1) and 250 g ethanol (30%) was thermal-extracted with reflux for one hour. After two extractions, an extract solution was formed and filtered through a 100mesh. After reduced pressure concentration and freeze-drying, a dried product was grounded to form powders.

### EXAMPLE 4

A solution containing 6.25 g Scutellaria baicalensis, 12.5 g Bupleurum chinense, 6.25 g Glycyrrhiza uralensis (a weight ratio of 1:2:1) and 250 g ethanol (30%) was thermal-extracted with reflux for one hour. After two extractions, an extract solution was formed and filtered through a 100mesh. After reduced pressure concentration and freeze-drying, a dried product was grounded to form powders.

### EXAMPLE 5

A solution containing 6.25 g Scutellaria baicalensis, 6.25 g Bupleurum chinense, 12.5 g Glycyrrhiza uralensis (a weight ratio of 1:1:2) and 250 g ethanol (30%) was thermal-extracted with reflux for one hour. After two extractions, an extract solution was formed and filtered through a 100mesh. After reduced pressure concentration and freeze-drying, a dried product was grounded to form powders.

### EXAMPLE 6

A solution containing 5 g Scutellaria baicalensis, 5 g Bupleurum chinense, 5 g Glycyrrhiza uralensis, 5 g Paeonia lactiflora, 5 g Schisandra chinensis (a weight ratio of 1:1:1:1:1) and 250 g water was thermal-extracted with reflux for one hour. After two extractions, an extract solution was formed and filtered through a 100mesh. After reduced pressure concentration and freeze-drying, a dried product was grounded to form powders.

### EXAMPLE 7

A solution containing 5 g Scutellaria baicalensis, 5 g Bupleurum chinense, 5 g Glycyrrhiza uralensis, 5 g Paeonia lactiflora, 5 g Schisandra chinensis (a weight ratio of 1:1:1:1:1) and 250 g ethanol (50%) was thermal-extracted with reflux for one hour. After two extractions, an extract solution was formed and filtered through a 100mesh. After reduced pressure concentration and freeze-drying, a dried product was grounded to form powders.

### EXAMPLE 8

A solution containing 5 g Scutellaria baicalensis, 5 g Bupleurum chinense, 5 g Glycyrrhiza uralensis, 5 g Paeonia lactiflora, 5 g Schisandra chinensis (a weight ratio of 1:1:1:1:1) and 250 g ethanol (95%) was thermal-extracted with reflux for one hour. After two extractions, an extract solution was formed and filtered through a 100mesh. After reduced pressure concentration and freeze-drying, a dried product was grounded to form powders.

Cell Toxicity of Herbal Extract in Neutrophil and T Lymphocytes

### EXAMPLE 9

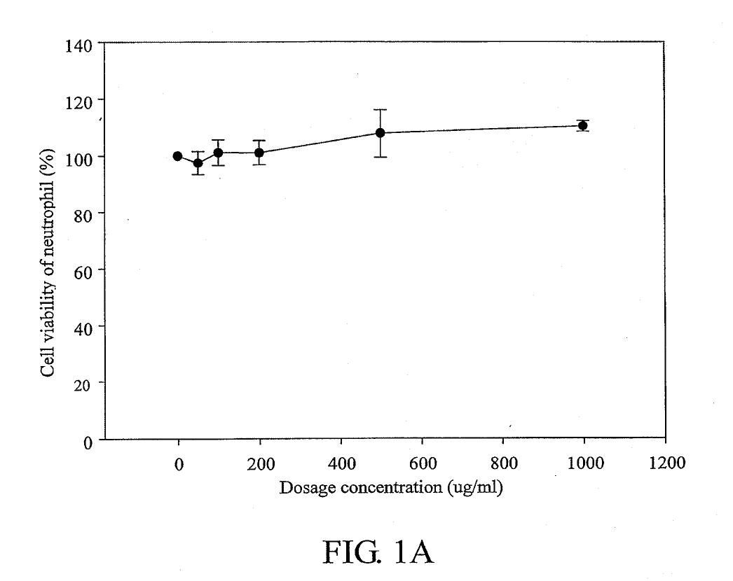

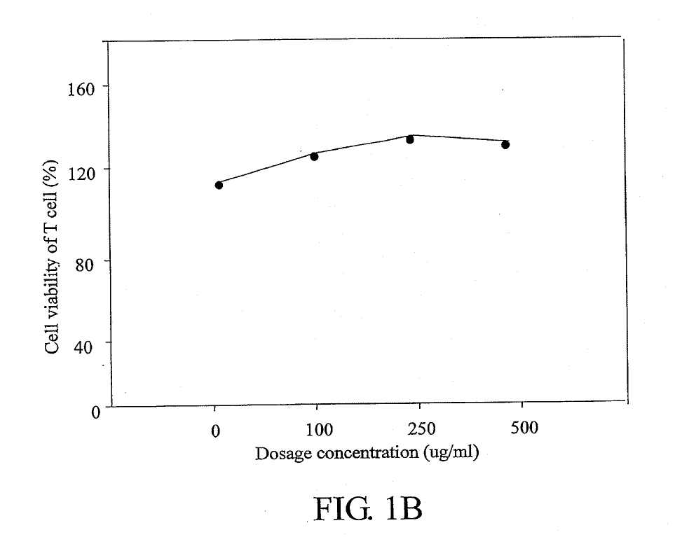

Cells were cultured in a 96-well plate. The herbal extract (prepared by Example 1) was then added. Next, 10 μL Alamarblue dye was added and placed in a culture box at 37° C. for 16 hours. The absorption values at 570 nm and 600 nm were measured, and are shown in FIGS. 1A and 1B. The results indicated that the cell toxicity of the herbal extract in neutrophil and T lymphocytes is non-obvious.

Test of Inducing Immune Cells to Produce Interferon by an Herbal Extract

### EXAMPLE 10

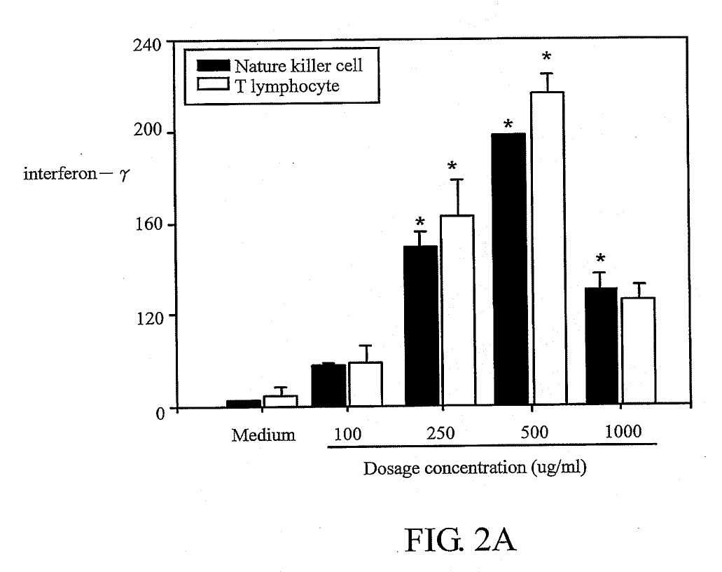

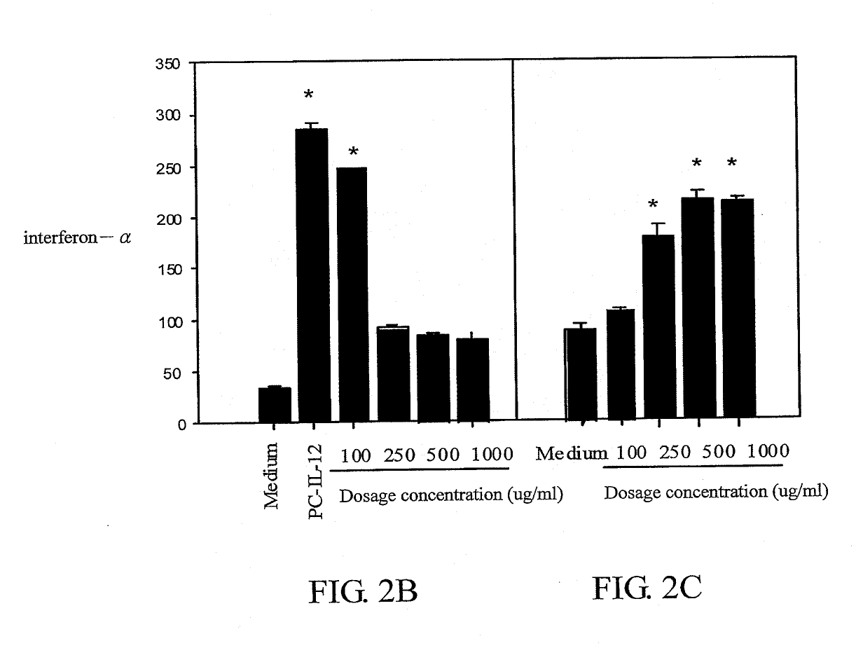

The herbal extract (prepared by Example 1) was added in T lymphocytes and natural killer cells (NK92) separated from peripheral blood. After 24 hours, the upper-layered solution was collected and the quantity of interferon thereof was measured by ELISA. The results indicated that the quantity of interferon-γ increased as the concentration of the herbal extract increased, exhibiting a dosage effect, as shown in FIG. 2A. The dosage effect appears in both the T lymphocytes and natural killer cells (NK92). The quantity of interferon-α induced by PC-IL-12 (positive control) and the herbal extract was measured in the same manner. The results indicated that the quantity of interferon-α produced by inducing natural killer cells (NK92) by the herbal extract with 100 μg/mL is more than that produced by inducing natural killer cells (NK92) by the herbal extract with other concentrations. Additionally, the quantity of interferon-α produced by inducing T lymphocytes by the herbal extract with 250-1,000 μg/mL was obvious, as shown in FIGS. 2B and 2C.

### EXAMPLE 11

The inducing activity for interferon-γ of the herbal extract (prepared by Example 3) was tested. The results indicated that the immune cells were induced by the sample with 500 μg/mL to produce interferon-γ of 94.3±15.7 pg/mL (the immune cells were induced by IL-12 (positive control) with 40 ng/mL to produce interferon-γ of 50±2.04 pg/mL).

### EXAMPLE 12

The inducing activity for interferon-γ of the herbal extract (prepared by Example 5) was tested. The results indicated that the immune cells were induced by the sample with 500 μg/mL to produce interferon-γ of 129.1±8.5 pg/mL (the immune cells were induced by IL-12 (positive control) with 40 ng/mL to produce interferon-γ of 50±2.04 pg/mL).

### EXAMPLE 13

The inducing activity for interferon-γ of the herbal extract (prepared by Example 6) was tested. The results indicated that the immune cells were induced by the sample with 1,000 g/mL to produce interferon-γ of 95.2±15.7 pg/mL (the immune cells were induced by IL-12 (positive control) with 40 ng/mL to produce interferon-γ of 50±2.04 pg/mL).

### EXAMPLE 14

The inducing activity for interferon-γ of the herbal extract (prepared by Example 7) was tested. The results indicated that the immune cells were induced by the sample with 1,000 μg/mL to produce interferon-γ of 1,563.6±44.9 pg/mL (the immune cells were induced by IL-12 (positive control) with 40 ng/mL to produce interferon-γ of 50±2.04 pg/mL).

### EXAMPLE 15

The inducing activity for interferon-γ of the herbal extract (prepared by Example 8) was tested. The results indicated that the immune cells were induced by the sample with 500 μg/mL to produce interferon-γ of 80.2±24.6 pg/mL (the immune cells were induced by IL-12 (positive control) with 40 ng/mL to produce interferon-γ of 50±2.04 pg/mL).

Test of Inducing Rates to Produce Interferon by the Herbal Extract

### EXAMPLE 16

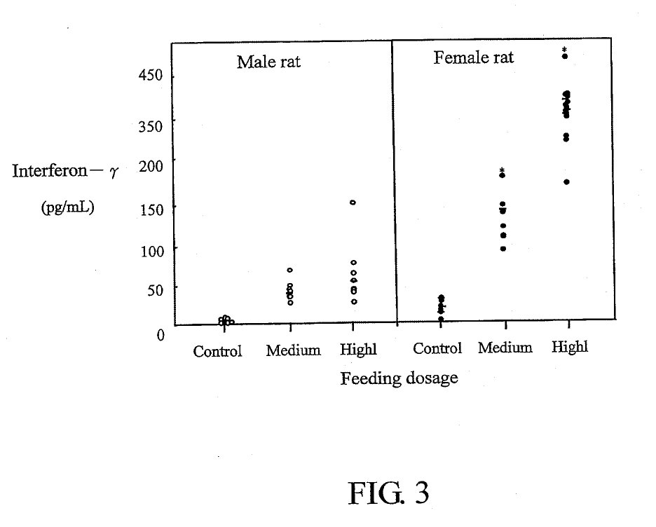

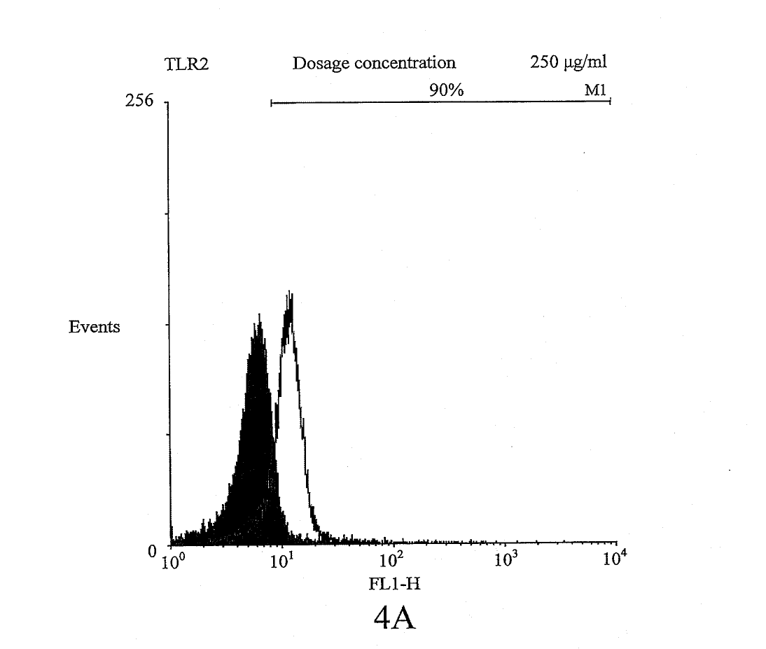

The inducing activity for interferon-γ in a living animal of the herbal extract (prepared by Example 1) was tested. After feeding (28 days), the quantity of interferon-γ in blood of Wistar rats (control: drinking water; medium: feeding the herbal extract of 5 g/kg; high: feeding the herbal extract of 10 g/kg) was measured on the 29^{th} day. The results indicated that the quantity of interferon-γ induced by the herbal extract in a subject was obvious, especially in female rats, as shown in FIG. 3.

Test of Expression of Toll-Like Receptors of PBMC/T Cells Activated by the Herbal Extract

### EXAMPLE 17

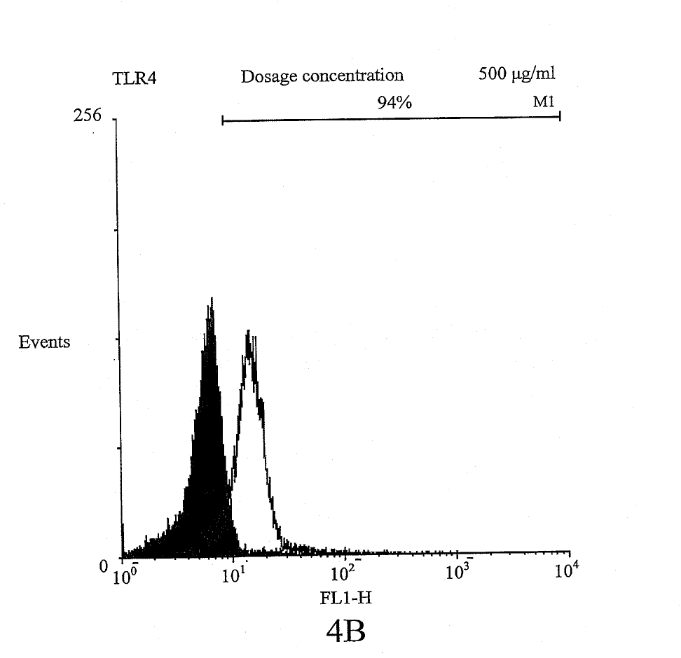

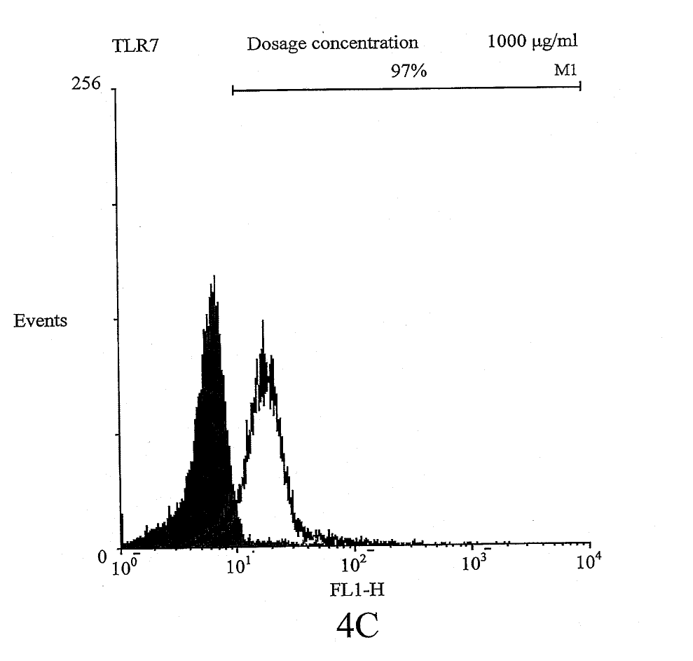

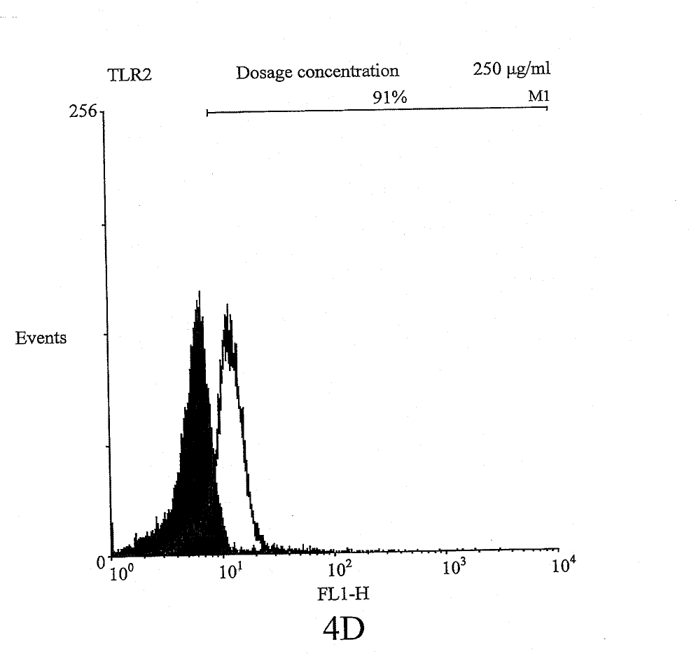

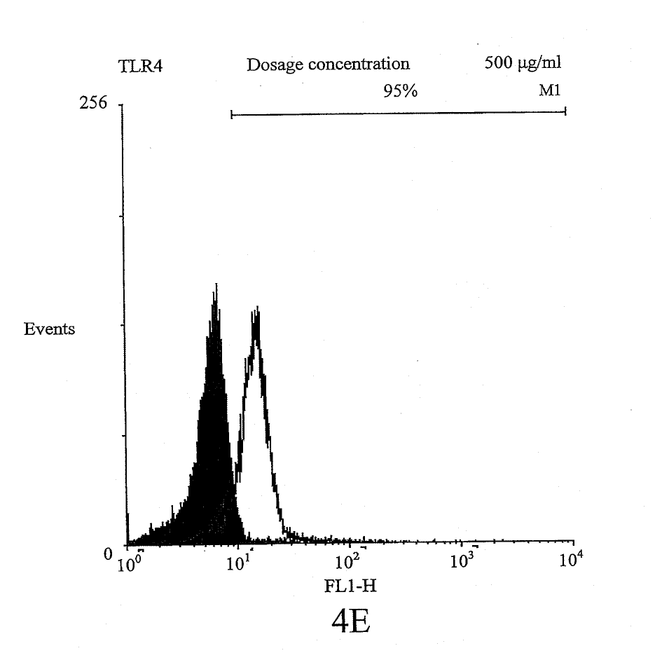

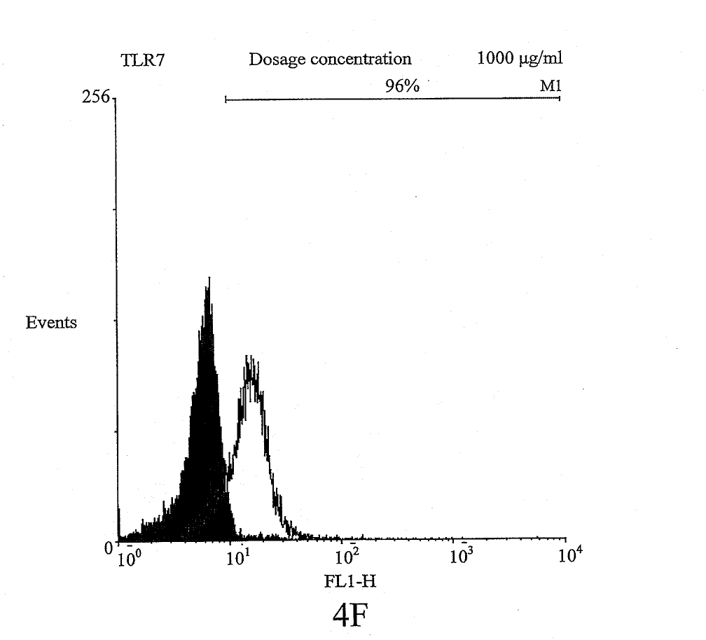

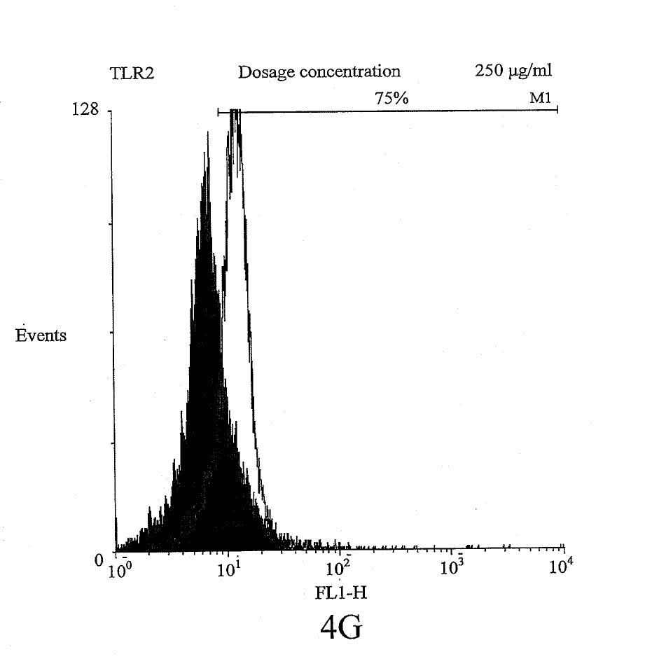

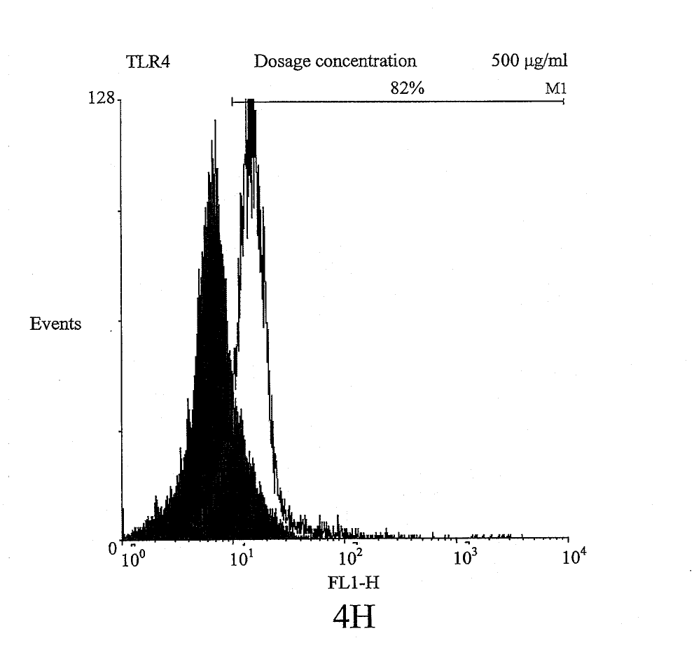

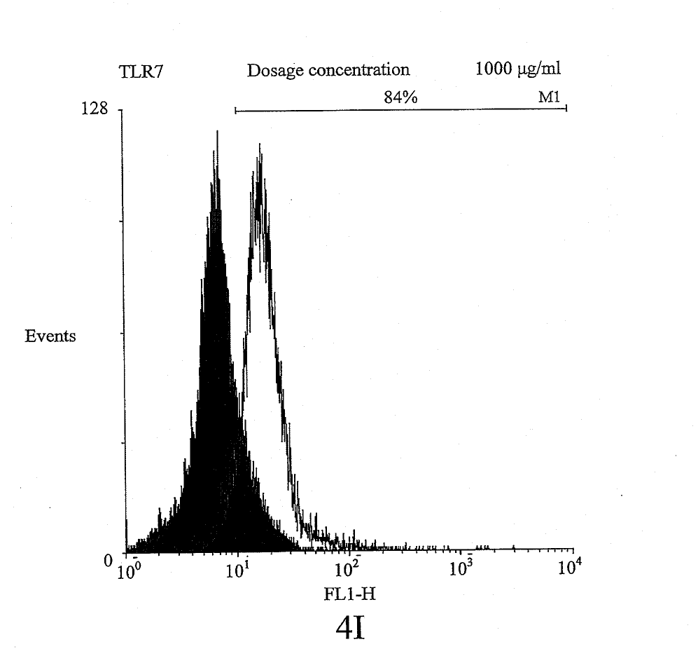

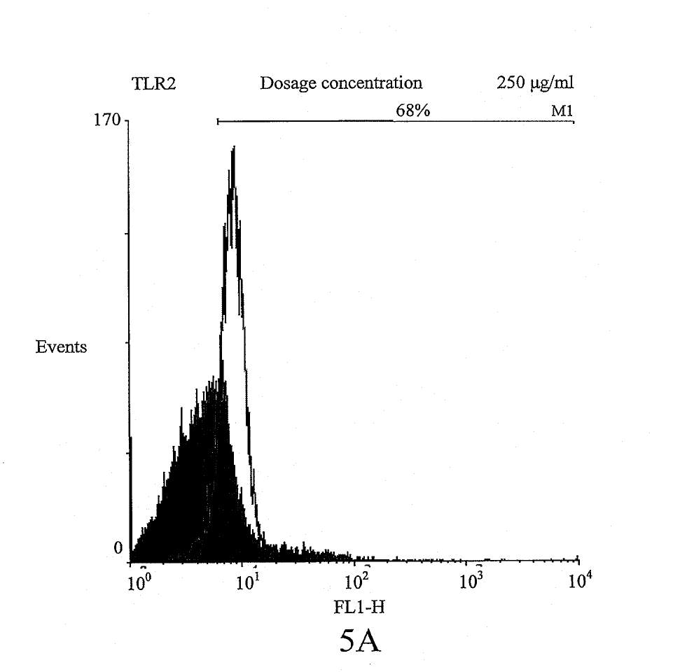

The expressions of Toll-like receptor 2 (TLR2), Toll-like receptor 4 (TLR4) and Toll-like receptor 7 (TLR7) activated by the herbal extract (prepared by Example 1) were tested. The cells were labeled by dyeing with TLR2, TLR4 and TLR7 antibodies and then analyzed by a fluorescence activated cell sorter (FACS). The results indicated that when a low dosage (250 μg/mL) of the herbal extract was applied, expressions of above 75% for the TLR2, TLR4 or TLR7 were achieved, as shown in FIGS. 4A-4I.

### EXAMPLE 18

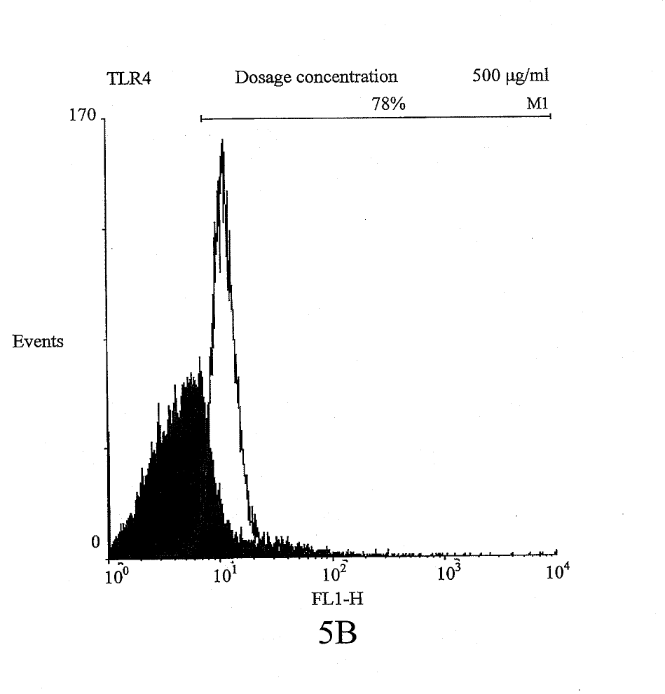

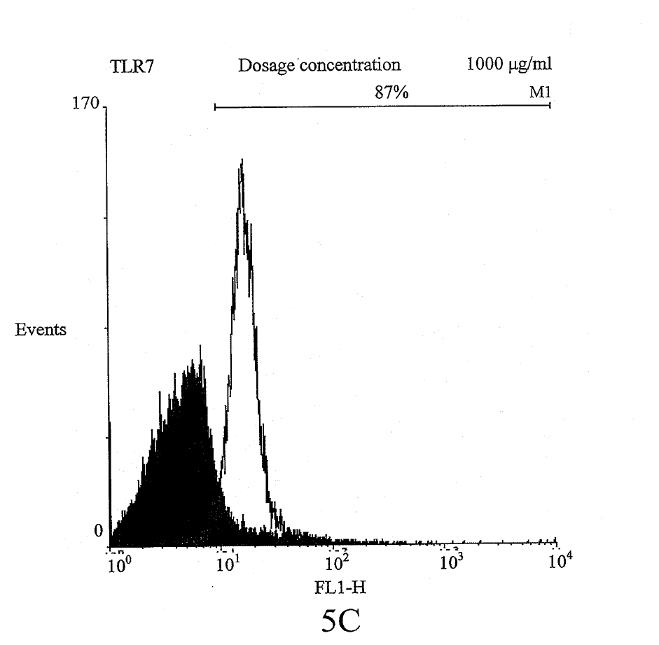

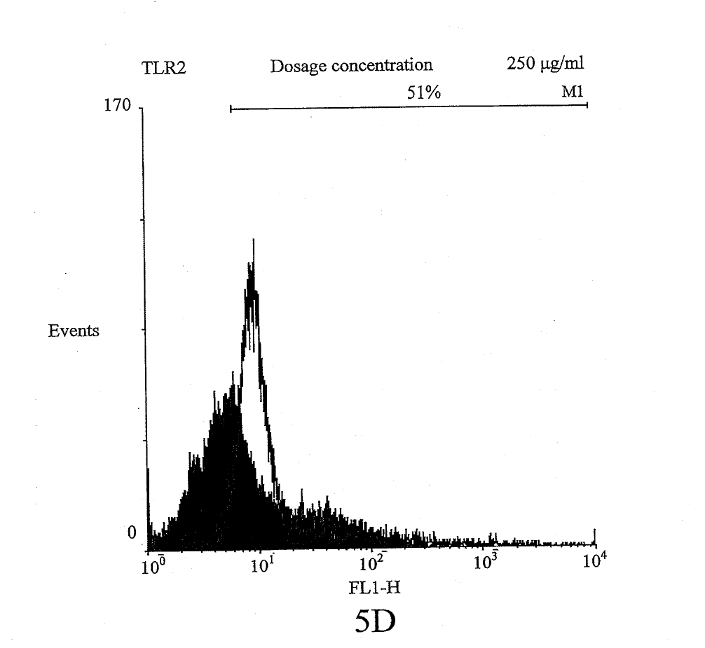

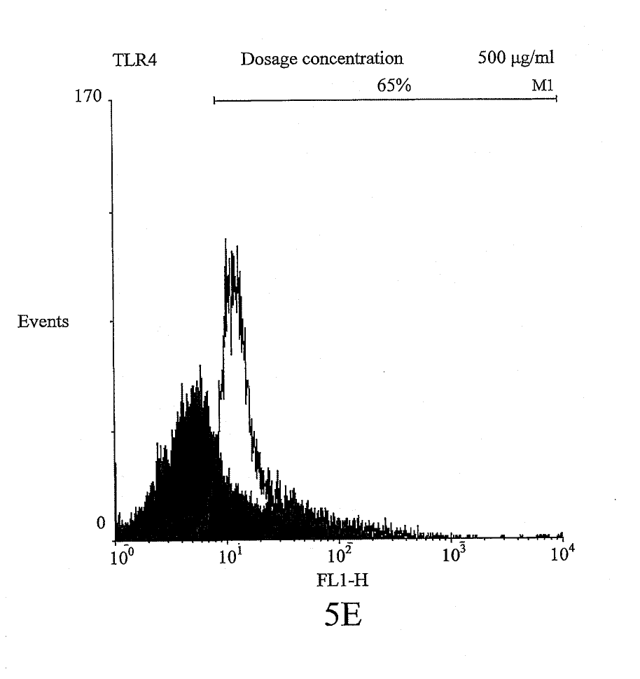

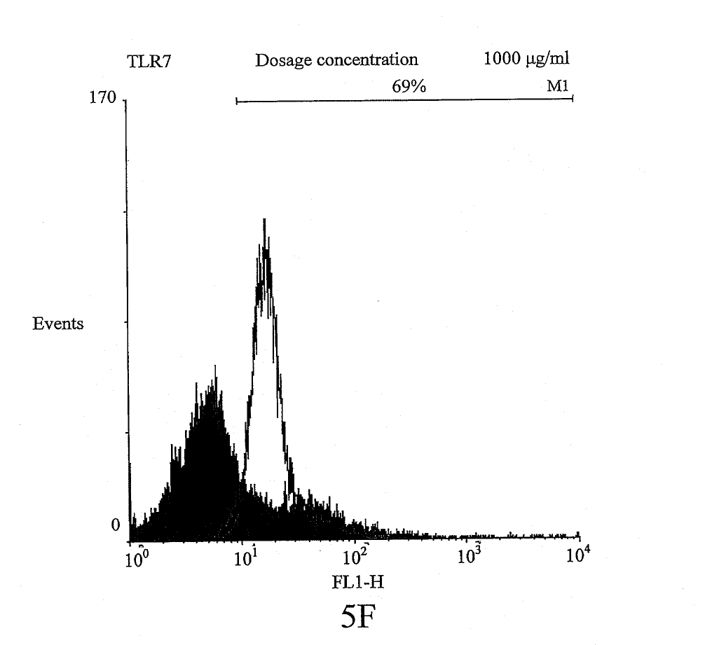

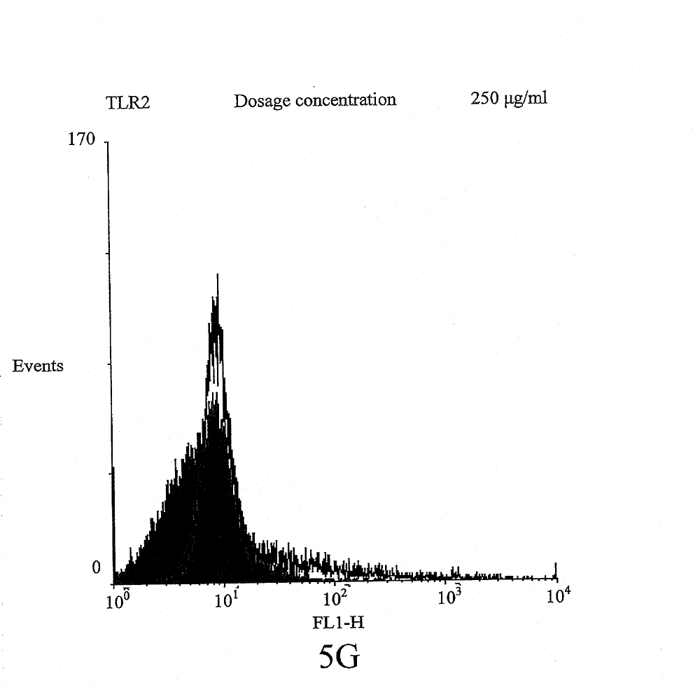

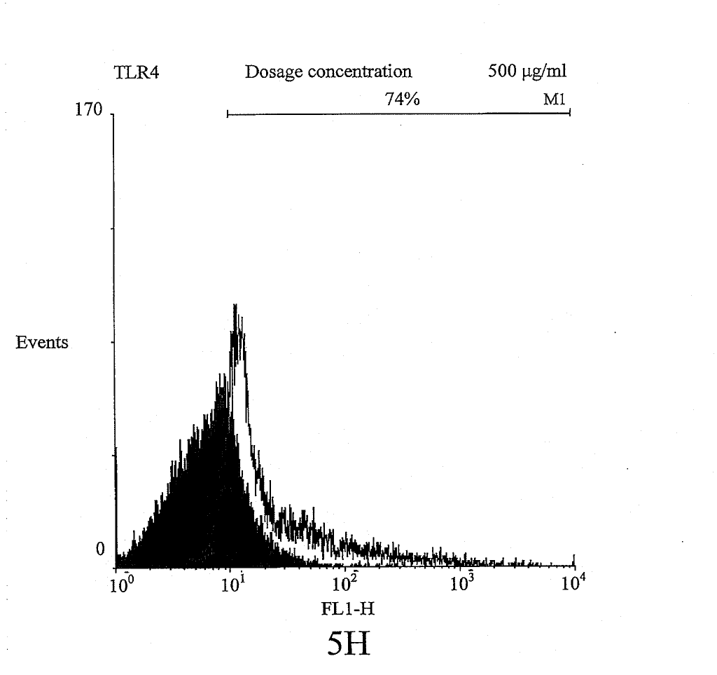

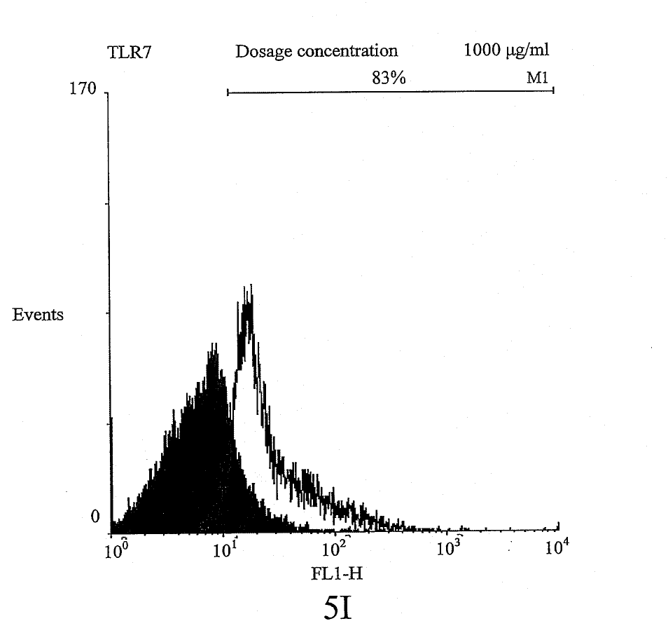

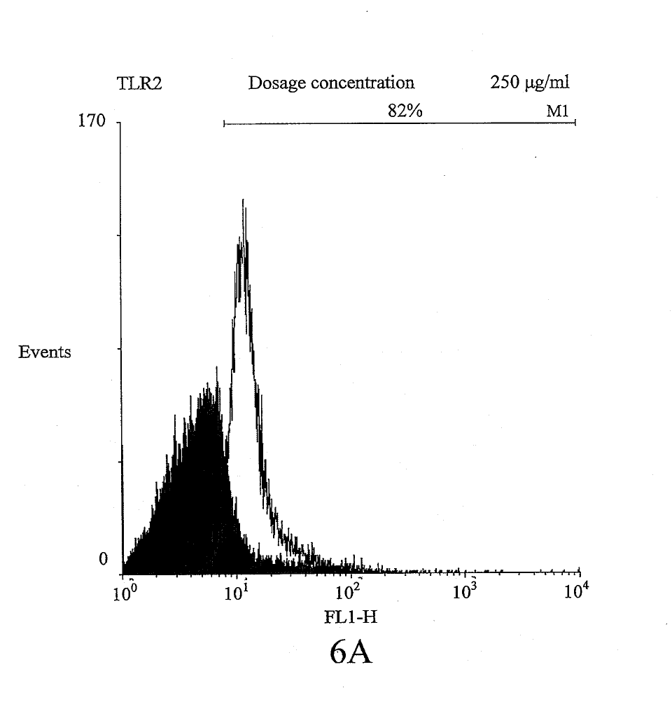

The expressions of Toll-like receptor 2 (TLR2), Toll-like receptor 4 (TLR4) and Toll-like receptor 7 (TLR7) activated by the herbal extract (prepared by Example 2) were tested. The results are shown in FIGS. 5A-5I. When the herbal extract of 500 μg/mL was applied, expressions of above 65% for the TLR2, TLR4 and TLR7 were achieved.

### EXAMPLE 19

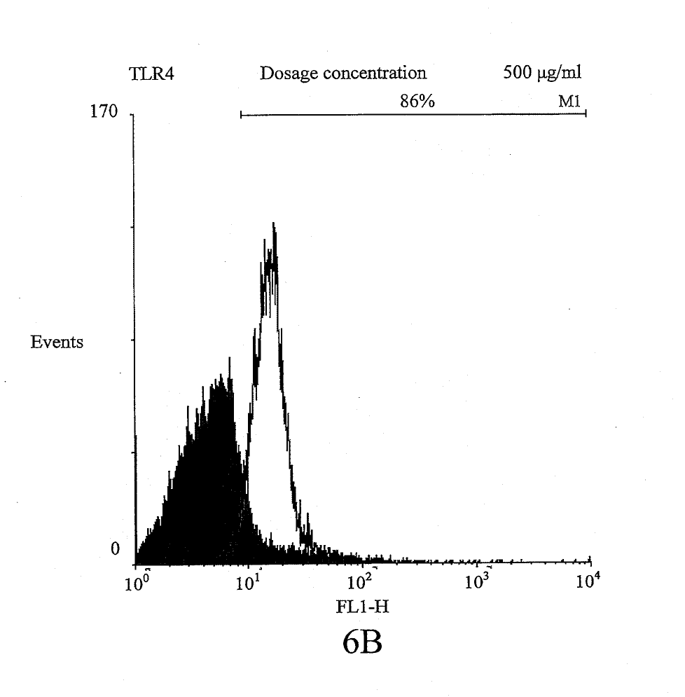

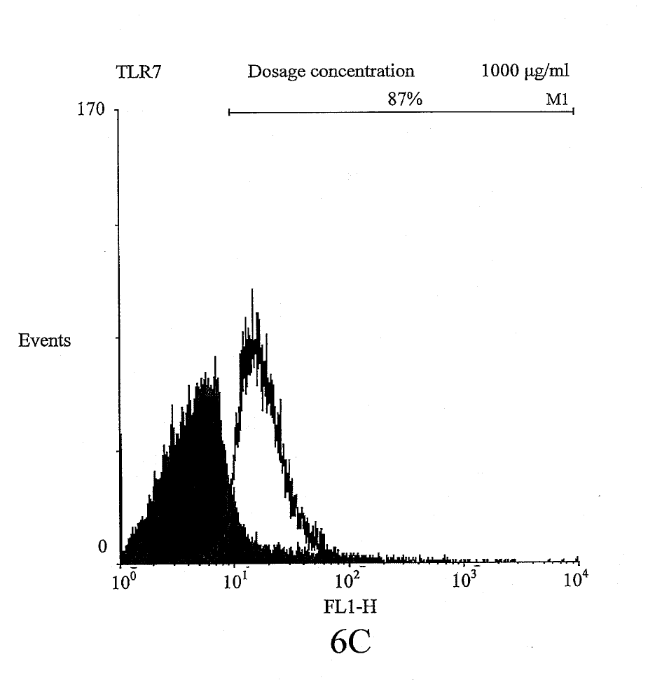

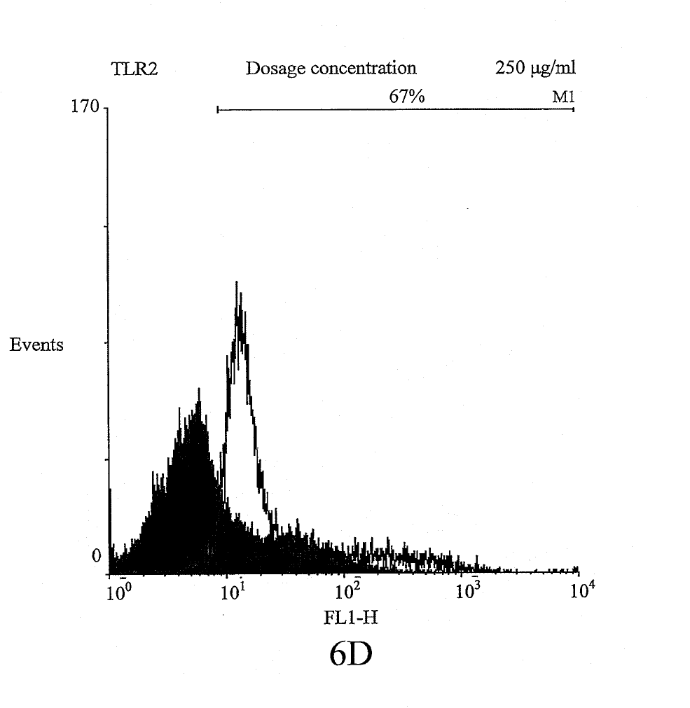

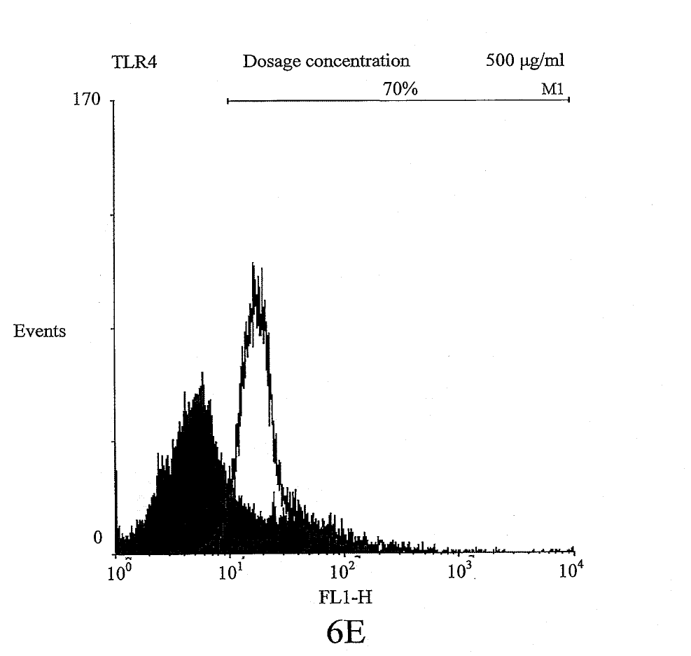

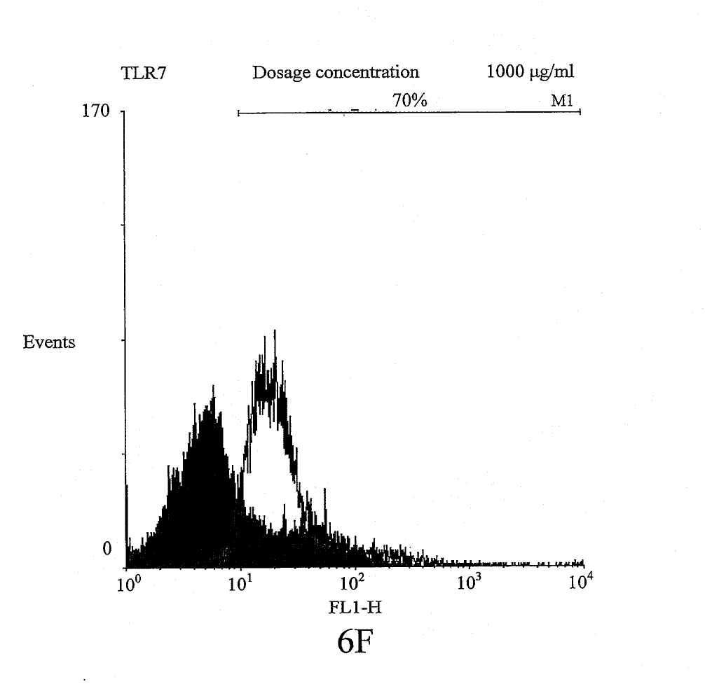

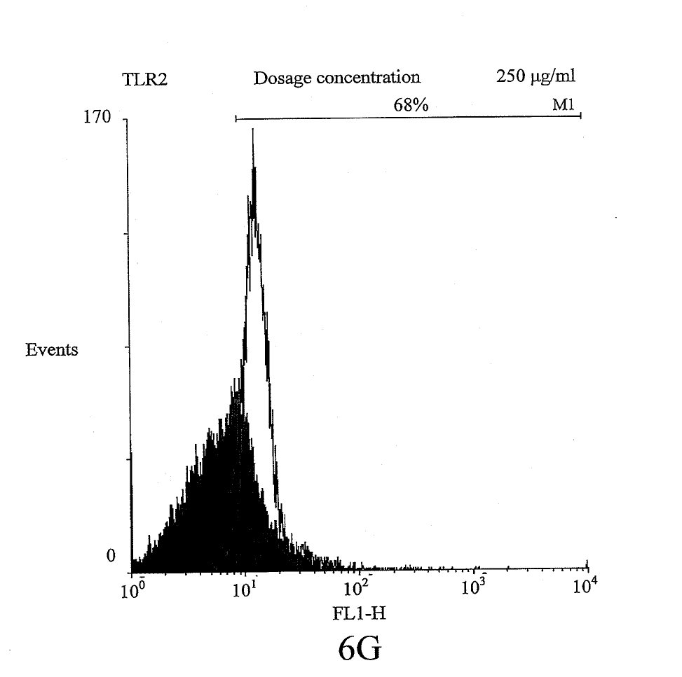

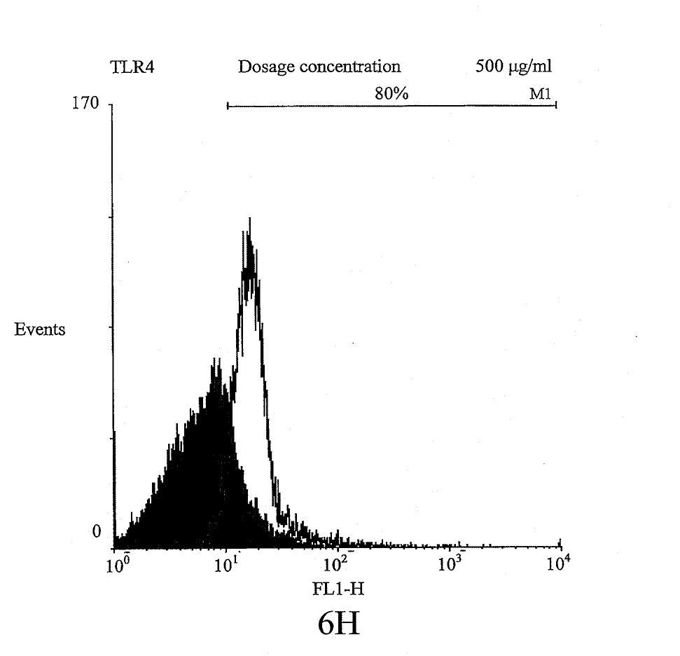

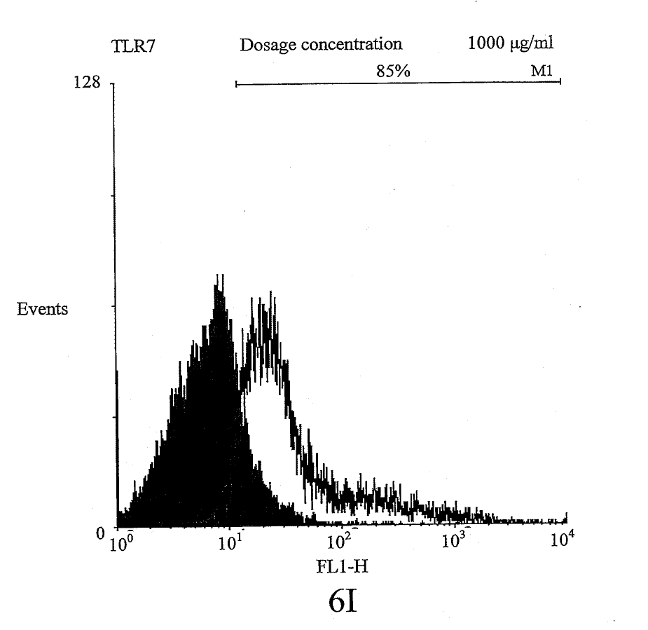

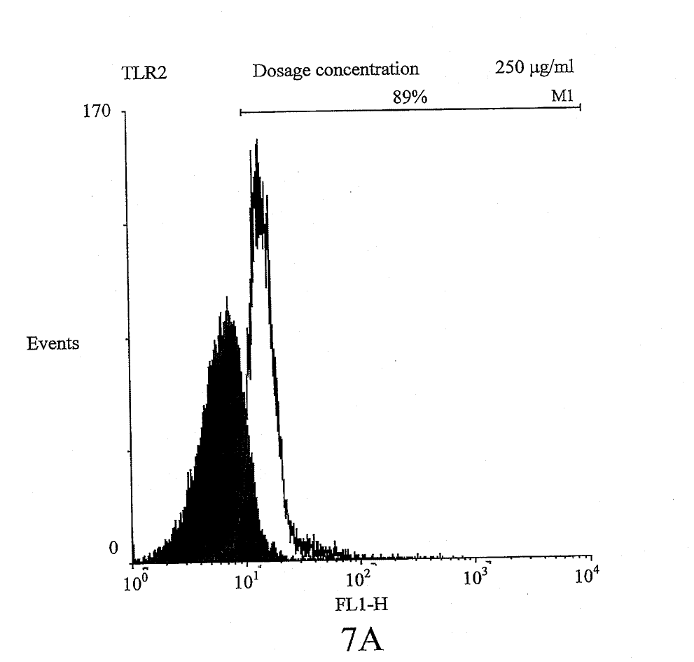

The expressions of Toll-like receptor 2 (TLR2), Toll-like receptor 4 (TLR4) and Toll-like receptor 7 (TLR7) activated by the herbal extract (prepared by Example 3) were tested. The results are shown in FIGS. 6A-6I. When a low dosage (250 μg/mL) of the herbal extract was applied, expressions of above 60% for the TLR2, TLR4 and TLR7 were achieved.

### EXAMPLE 20

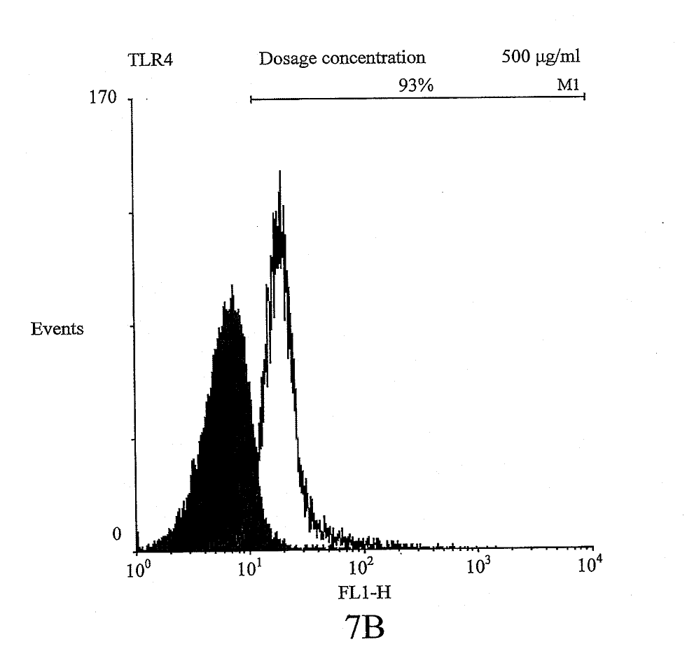

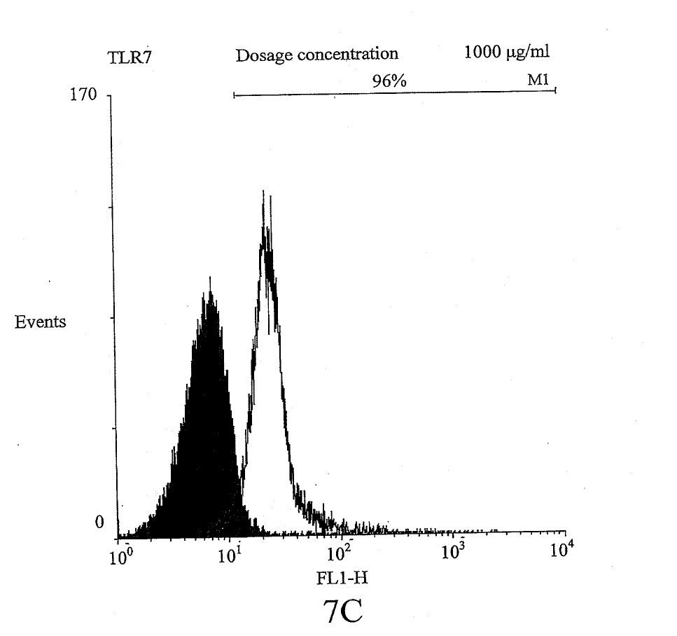

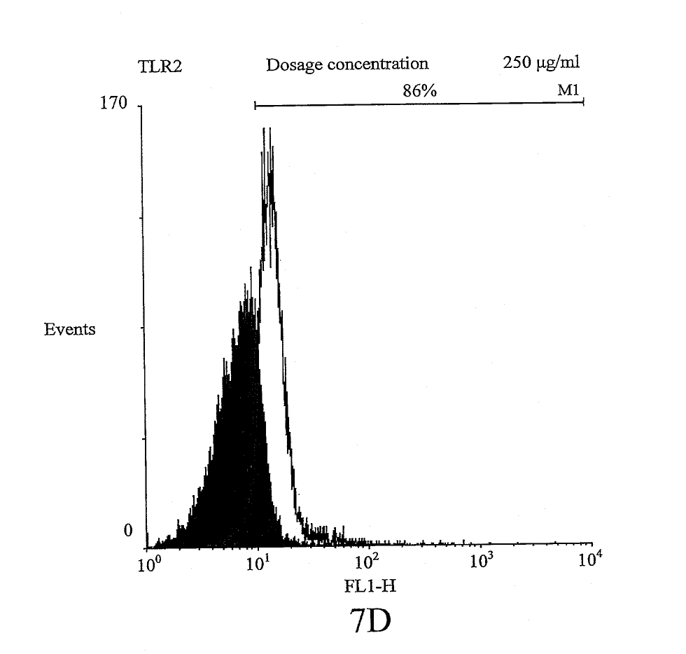

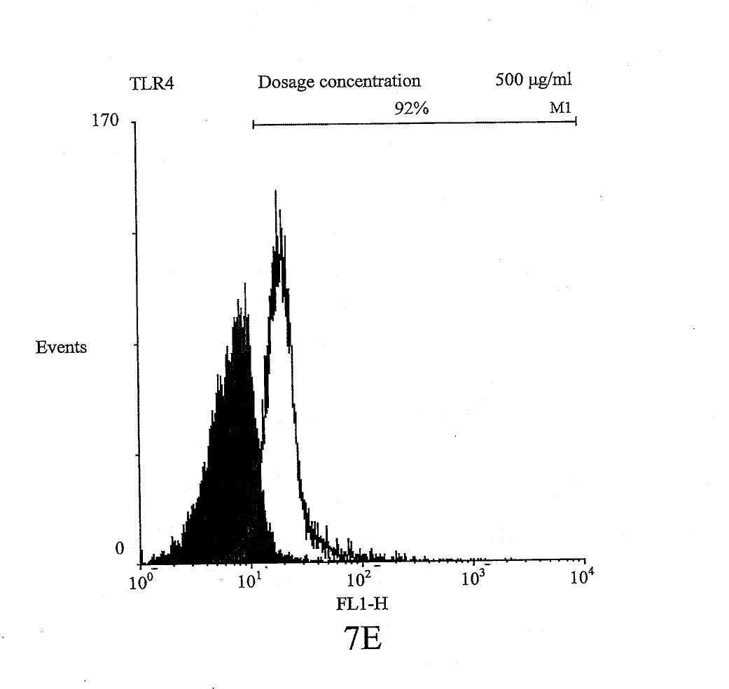

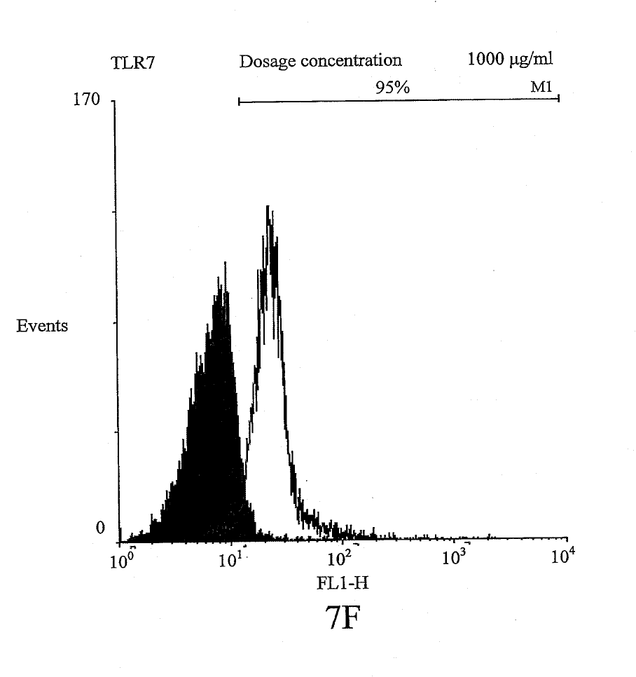

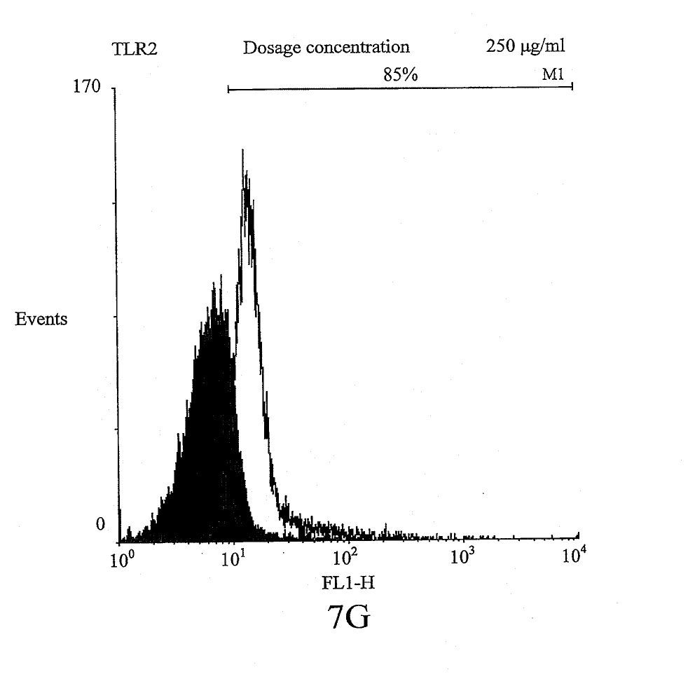

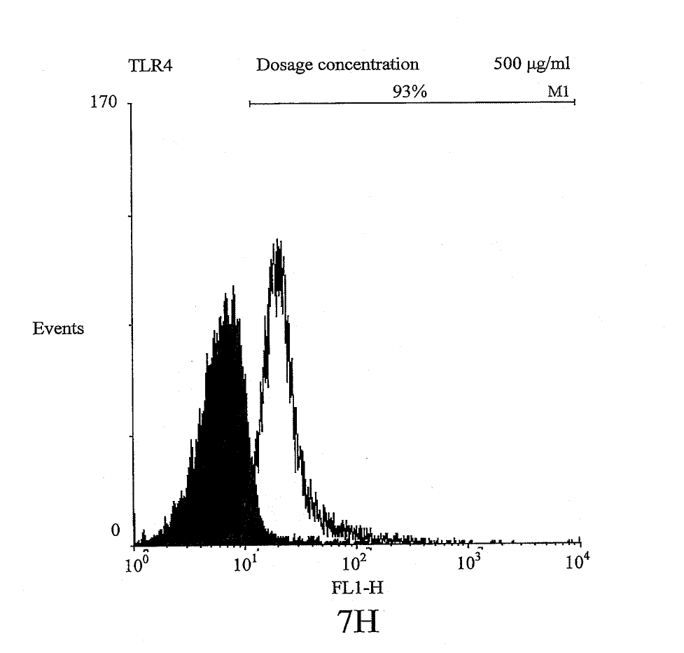

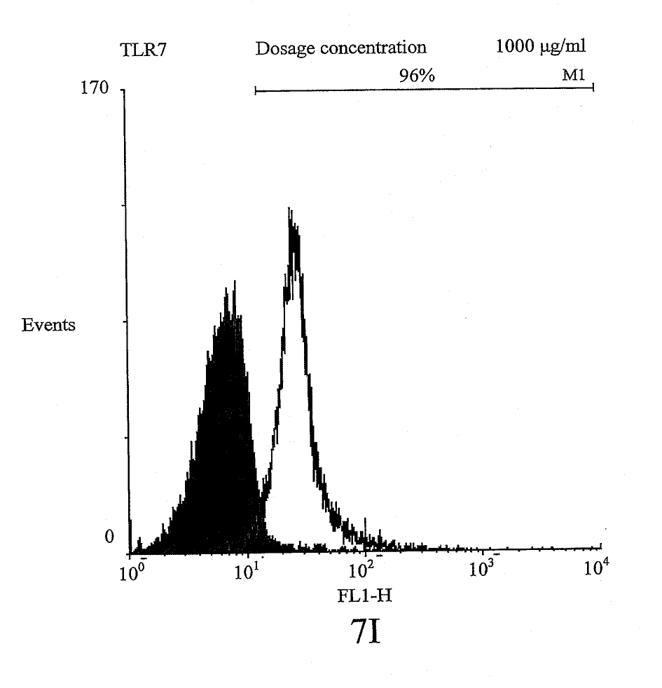

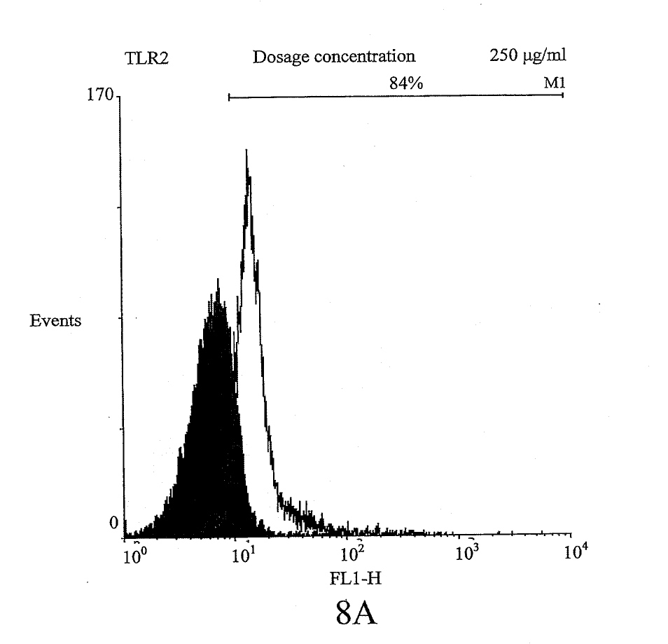

The expressions of Toll-like receptor 2 (TLR2), Toll-like receptor 4 (TLR4) and Toll-like receptor 7 (TLR7) activated by the herbal extract (prepared by Example 4) were tested. The results are shown in FIGS. 7A-7I. When a low dosage (250 μg/mL) of the herbal extract was applied, expressions of above 85% for the TLR2, TLR4 and TLR7 were achieved.

### EXAMPLE 21

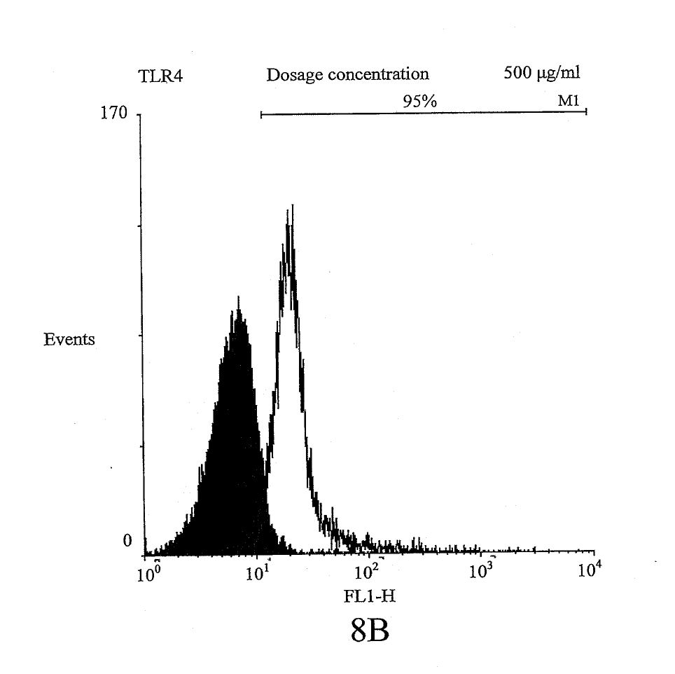

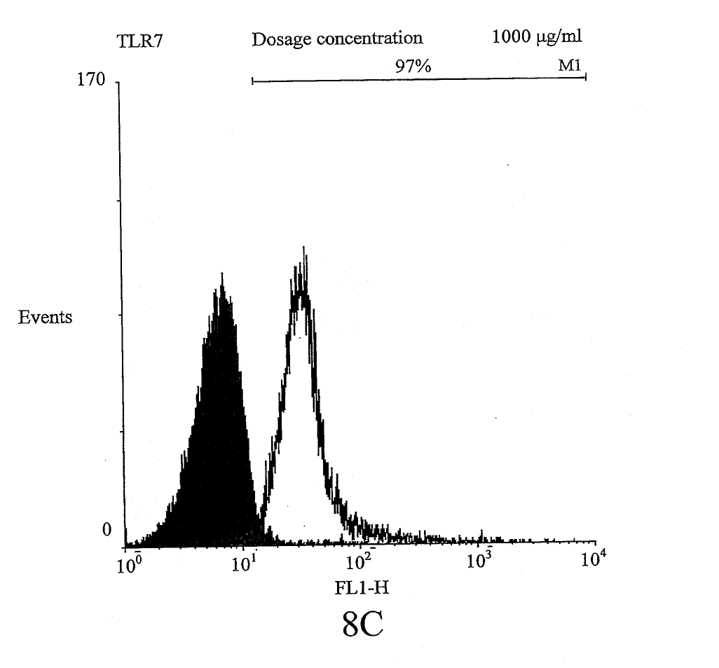

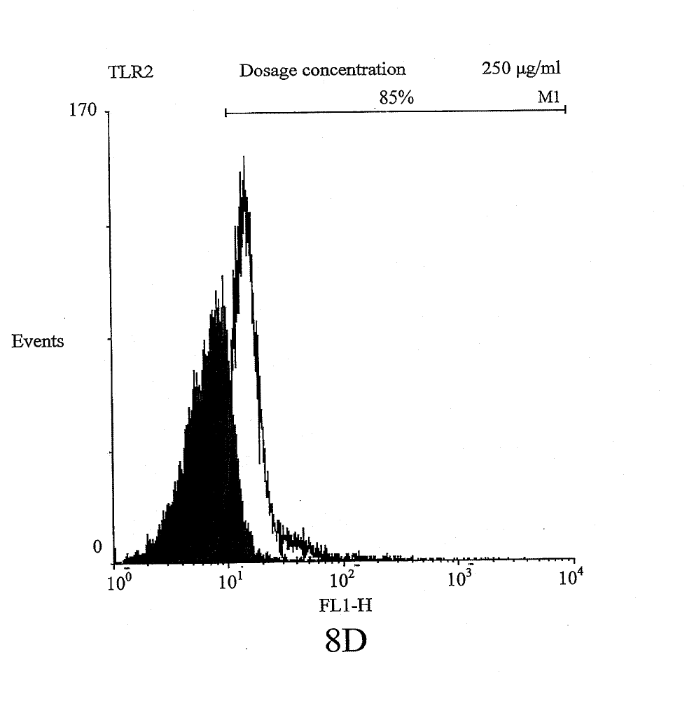

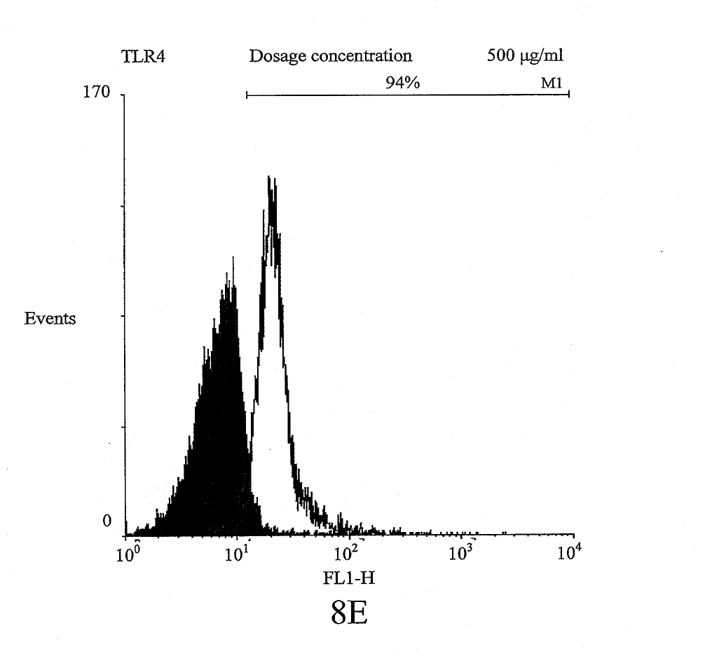

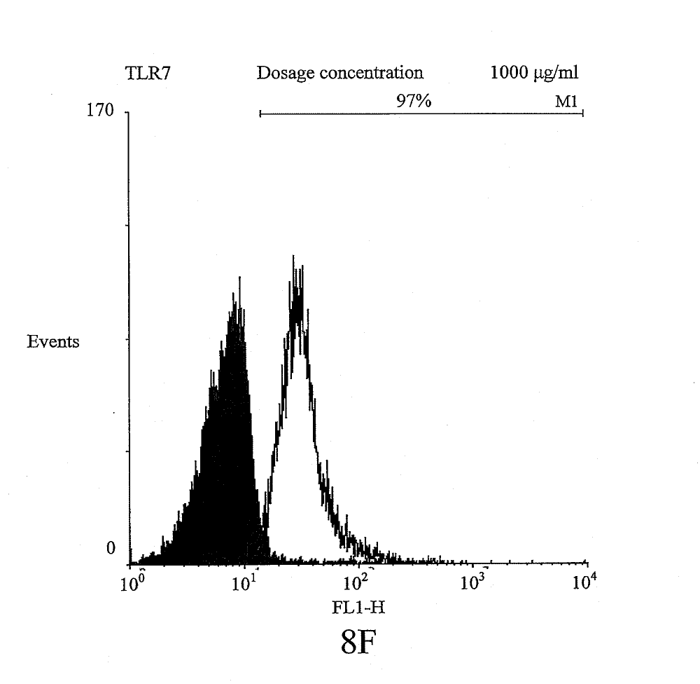

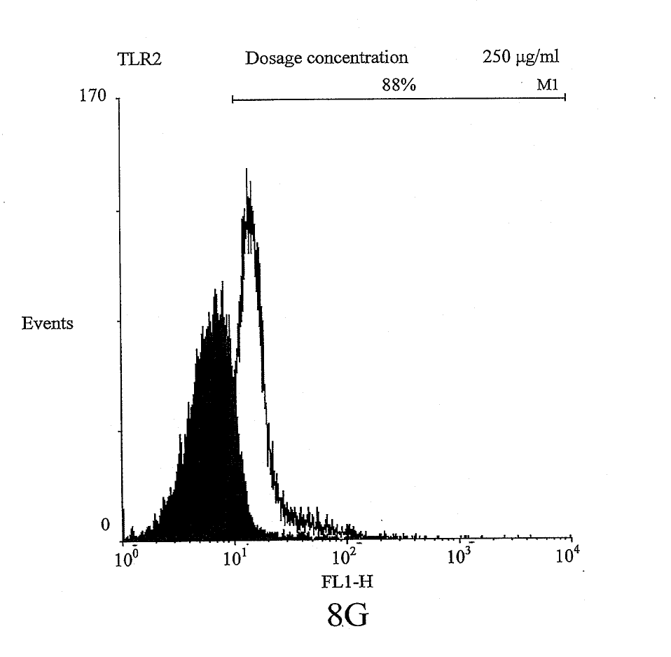

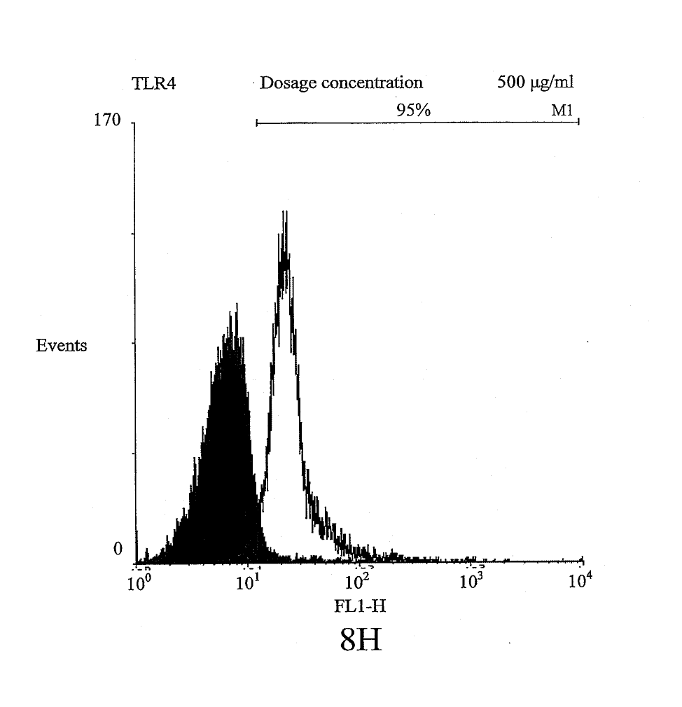

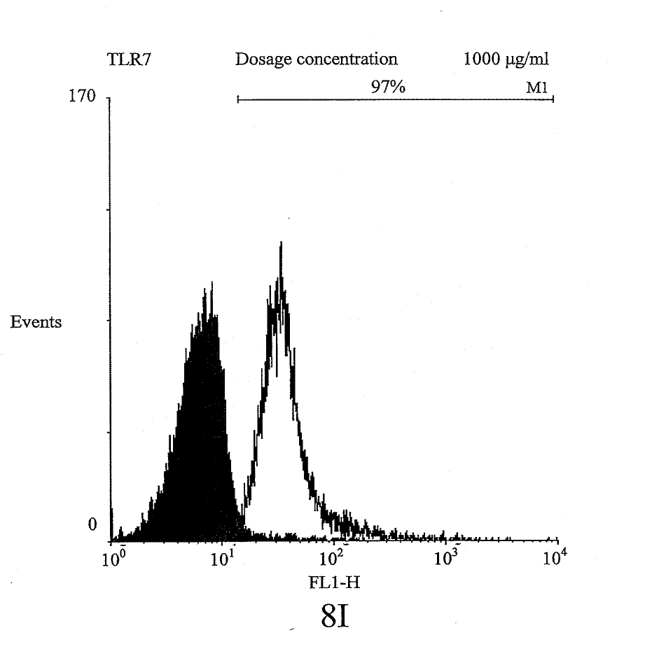

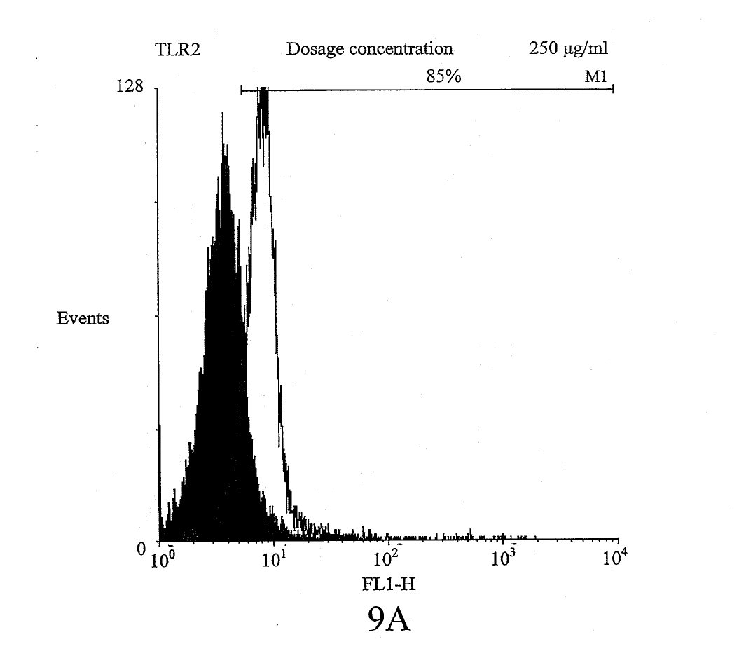

The expressions of Toll-like receptor 2 (TLR2), Toll-like receptor 4 (TLR4) and Toll-like receptor 7 (TLR7) activated by the herbal extract (prepared by Example 5) were tested. The results are shown in FIGS. 8A-8I. When a low dosage (250 μg/mL) of the herbal extract was applied, expressions of above 80% for the TLR2, TLR4 and TLR7 were achieved.

### EXAMPLE 22

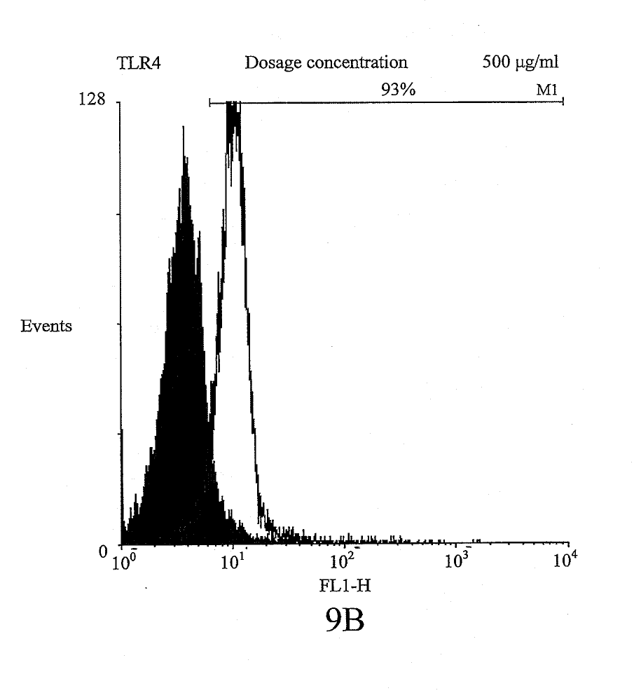

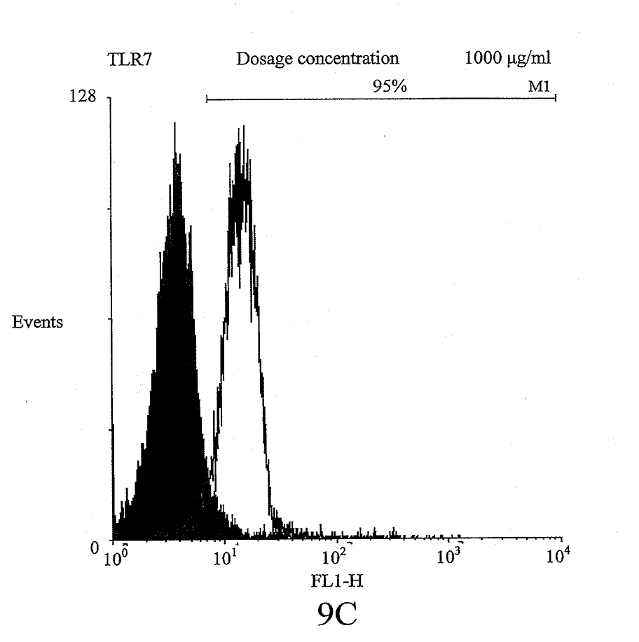

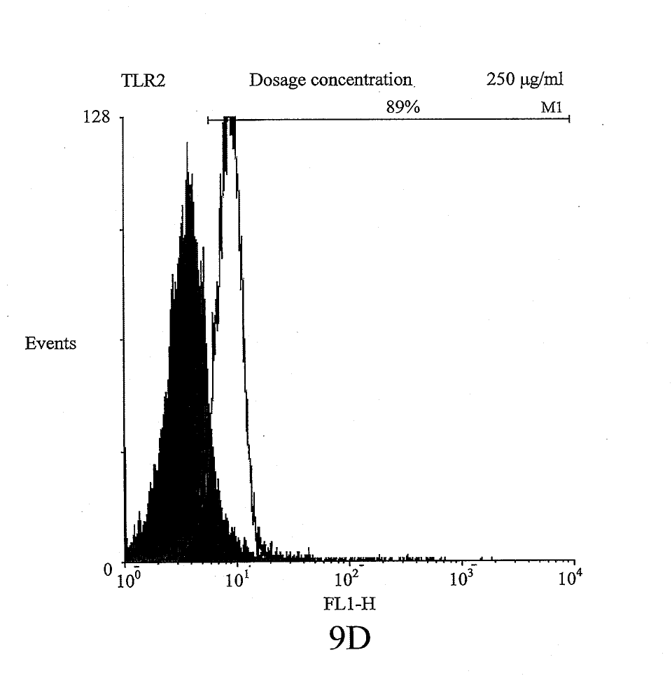

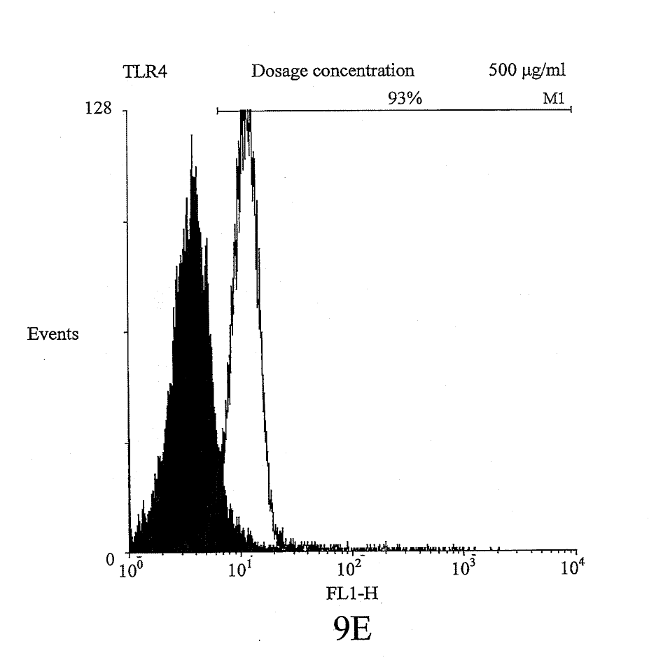

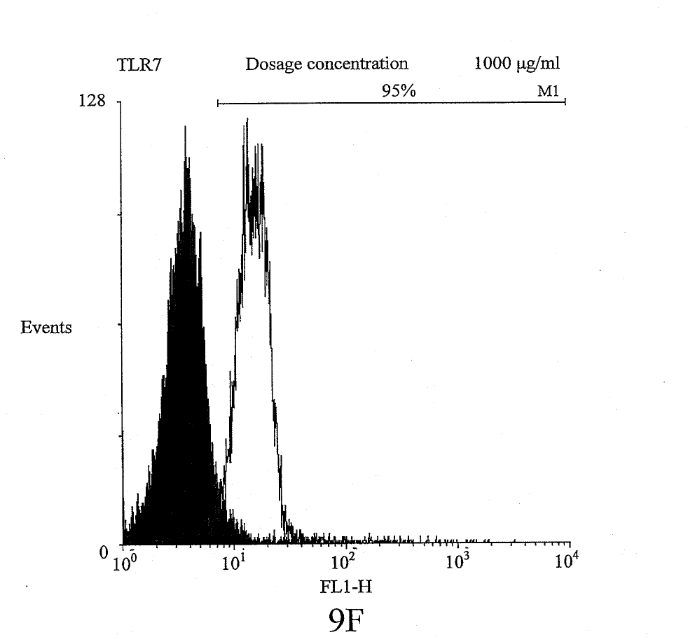

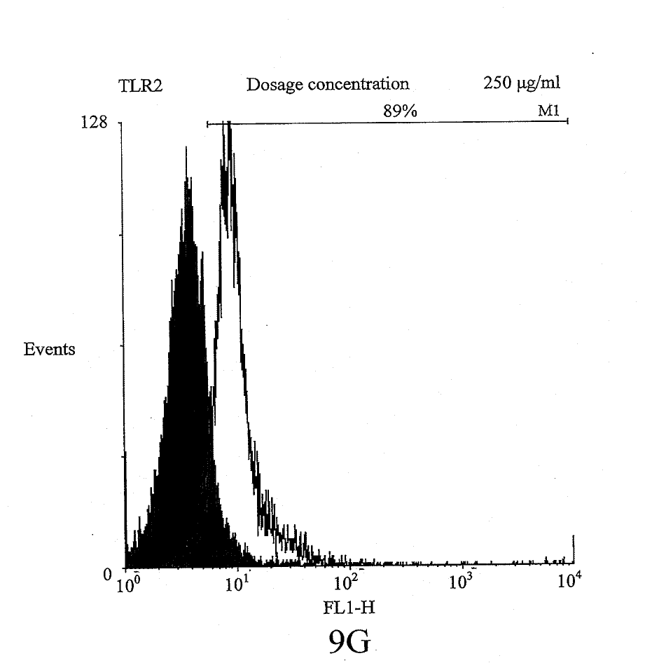

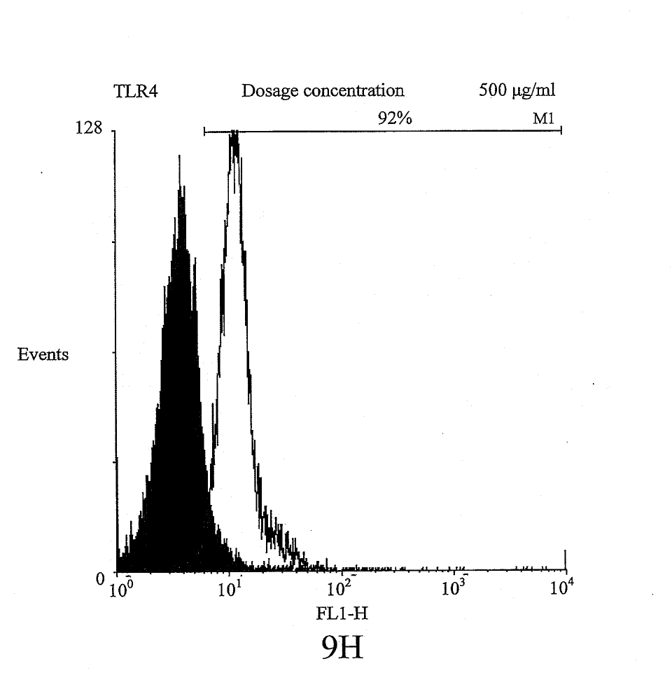

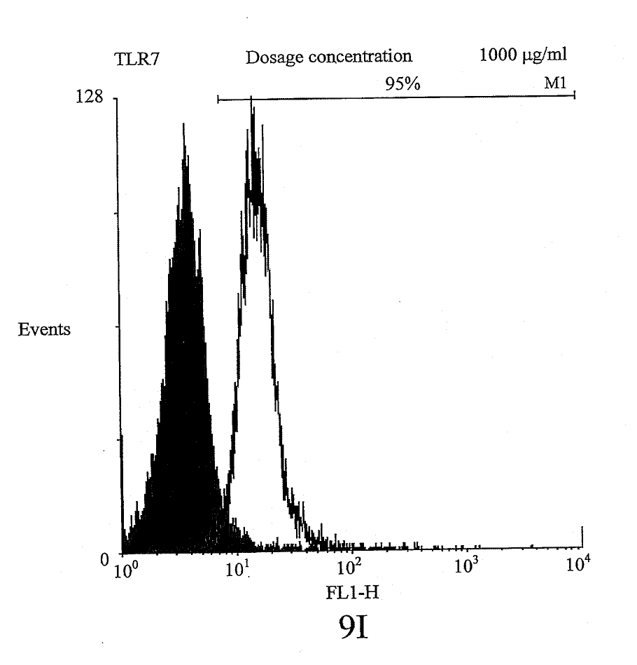

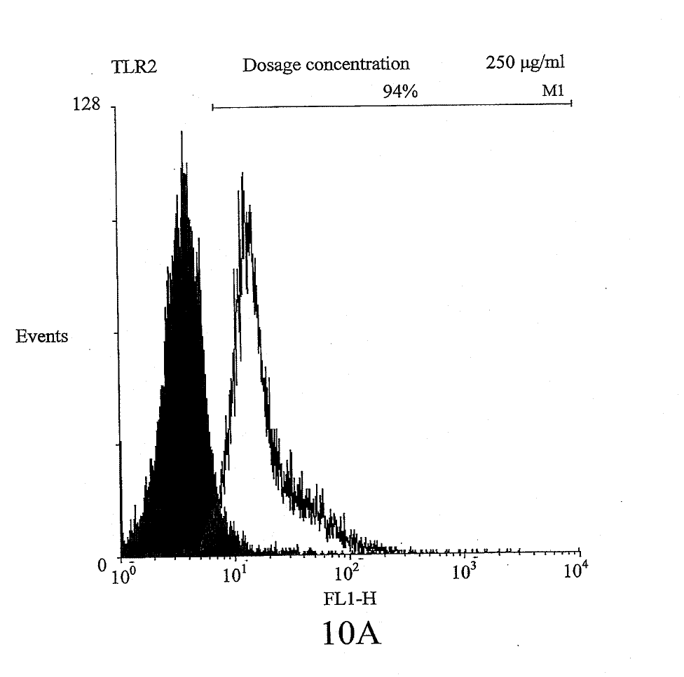

The expressions of Toll-like receptor 2 (TLR2), Toll-like receptor 4 (TLR4) and Toll-like receptor 7 (TLR7) activated by the herbal extract (prepared by Example 6) were tested. The results are shown in FIGS. 9A-9I. When a low dosage (250 μg/mL) of the herbal extract was applied, expressions of above 85% for the TLR2, TLR4 and TLR7 were achieved.

### EXAMPLE 23

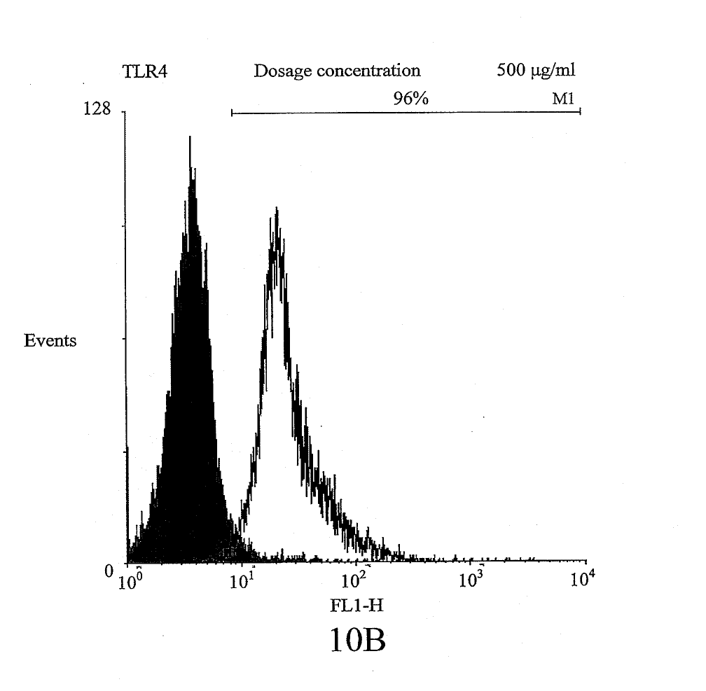

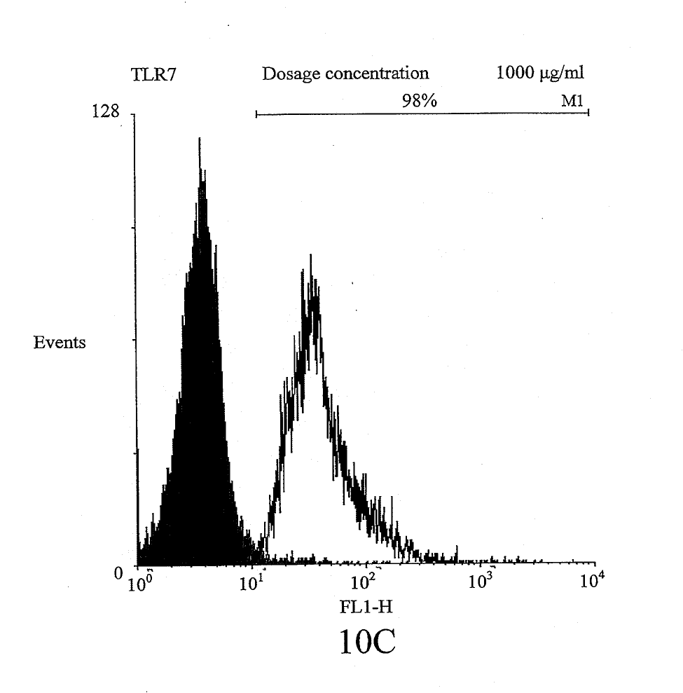

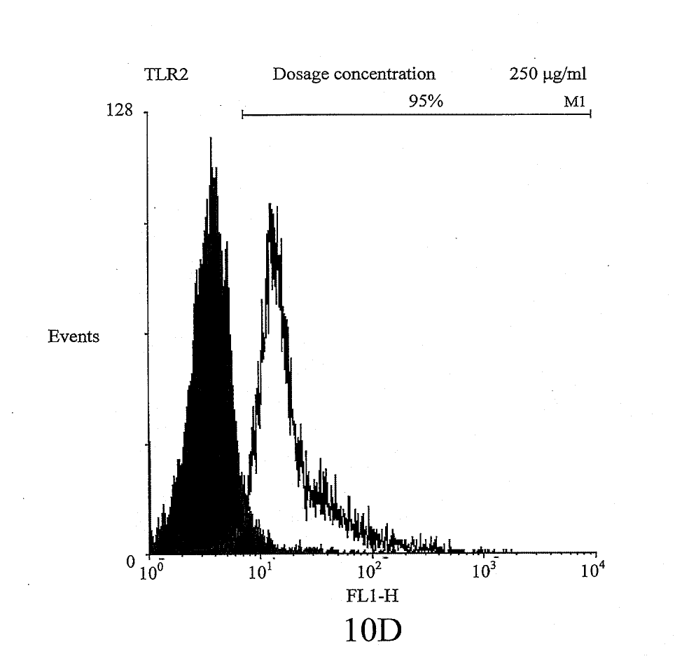

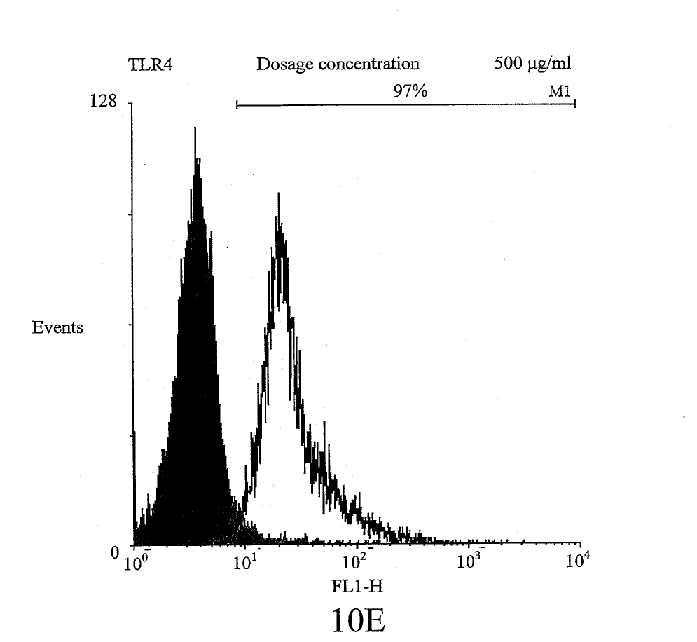

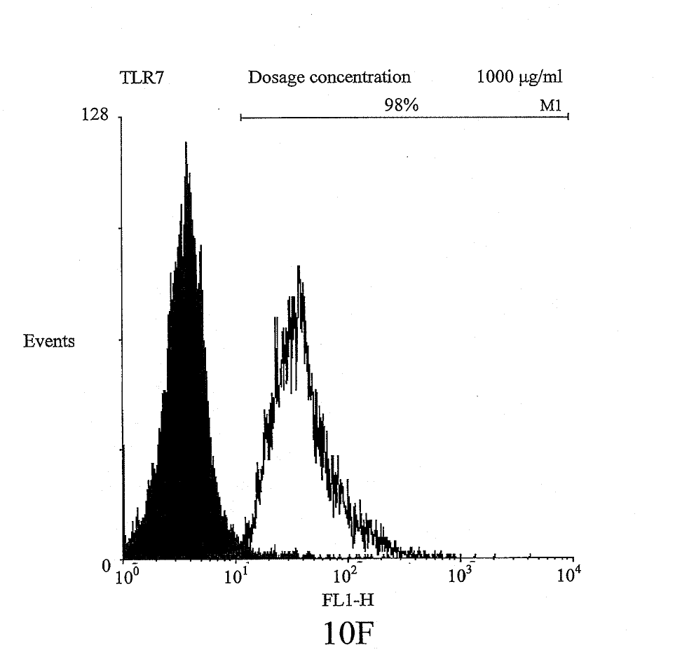

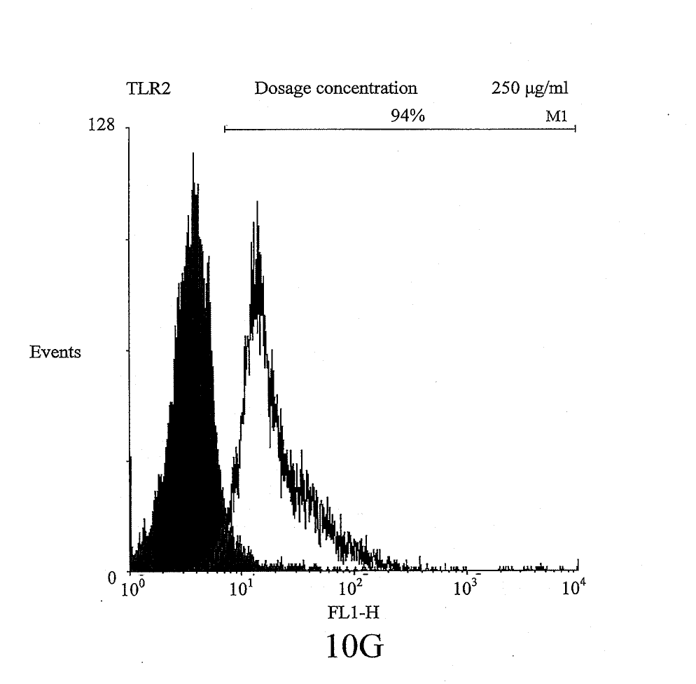

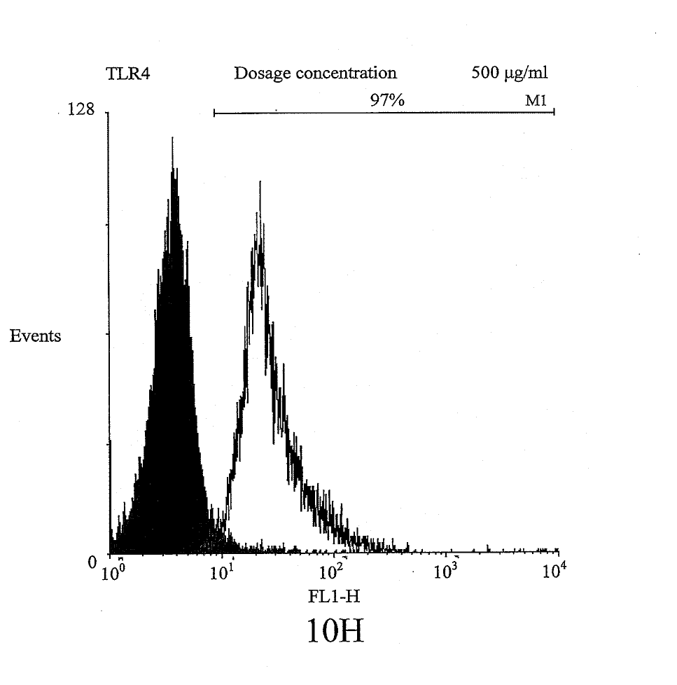

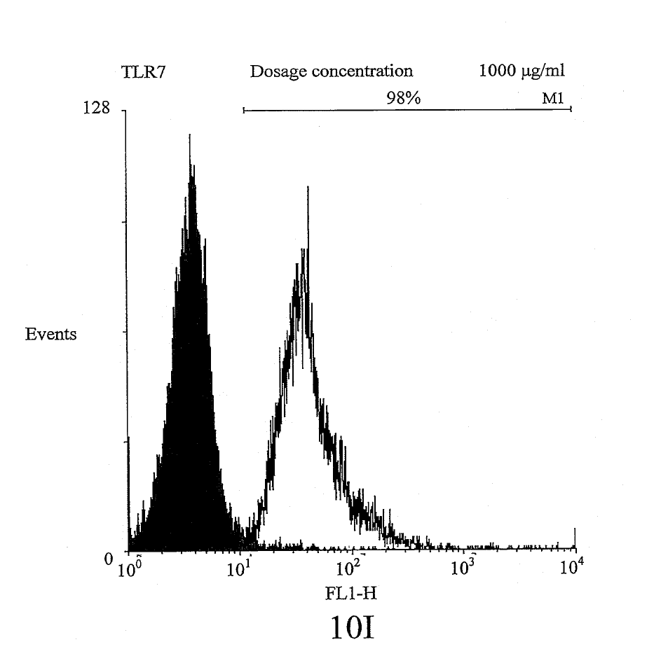

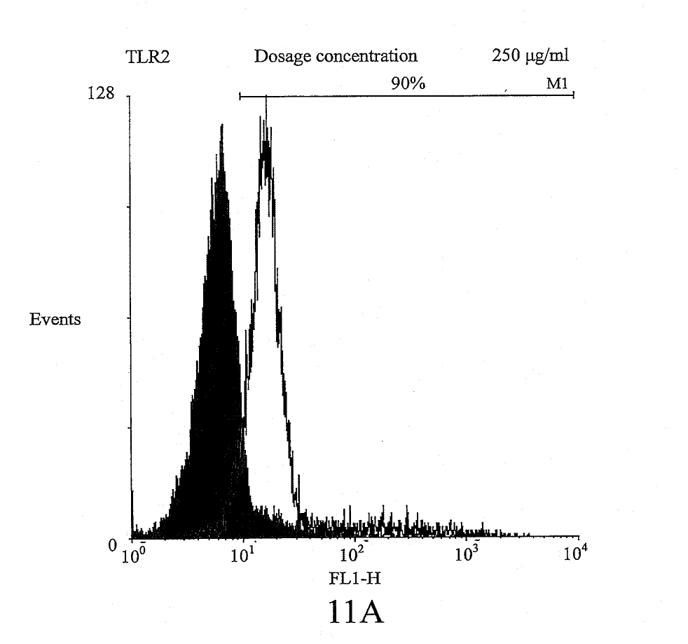

The expressions of Toll-like receptor 2 (TLR2), Toll-like receptor 4 (TLR4) and Toll-like receptor 7 (TLR7) activated by the herbal extract (prepared by Example 7) were tested. The results are shown in FIGS. 10A-10I. When a low dosage (250 μg/mL) of the herbal extract was applied, expressions of above 94% for the TLR2, TLR4 and TLR7 were achieved.

### EXAMPLE 24

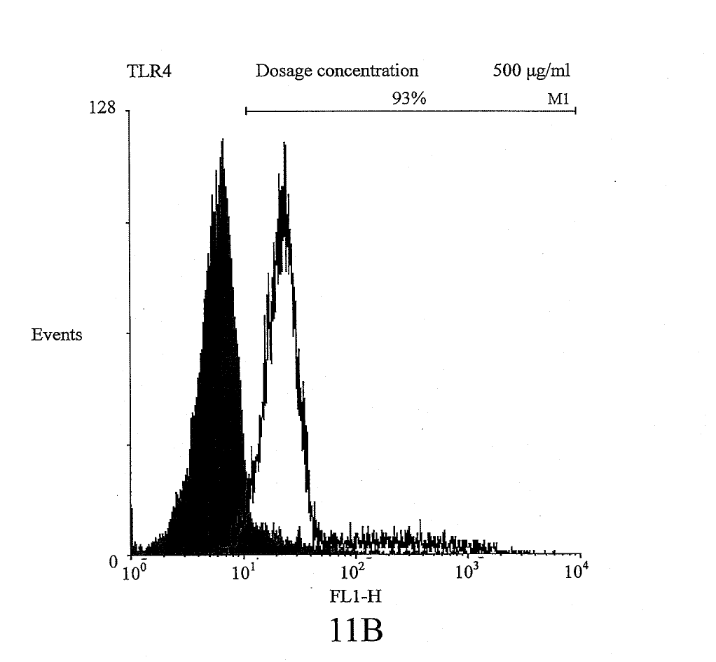

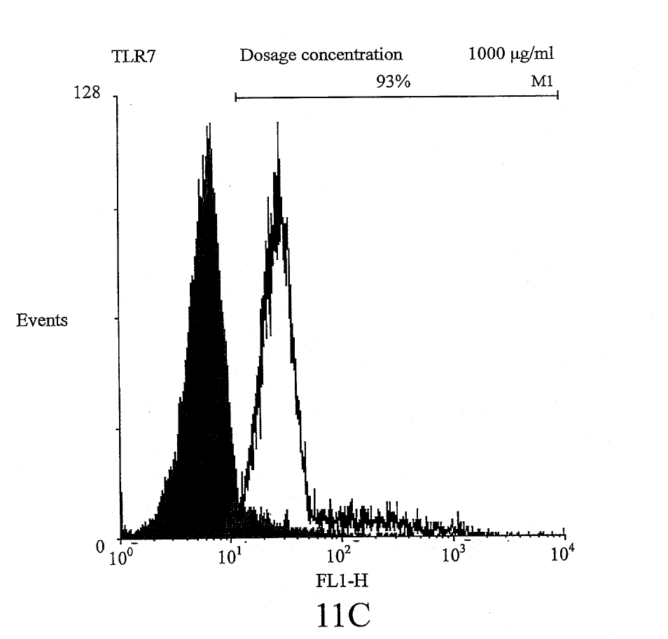

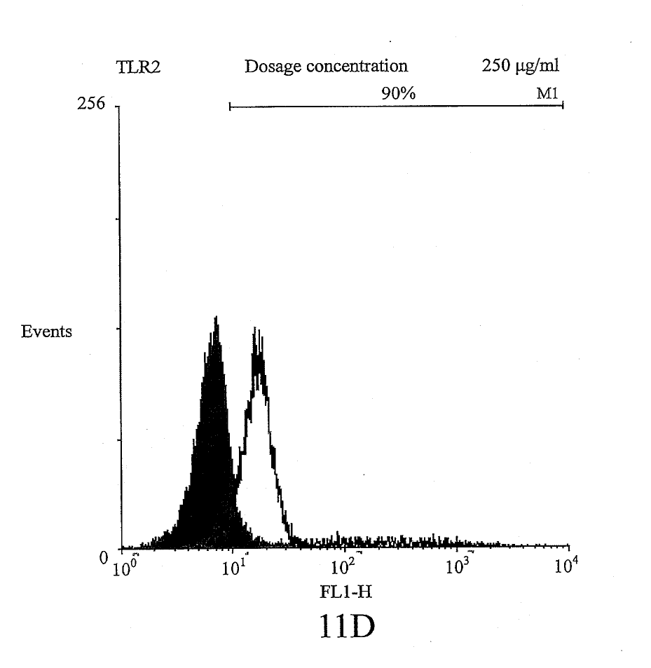

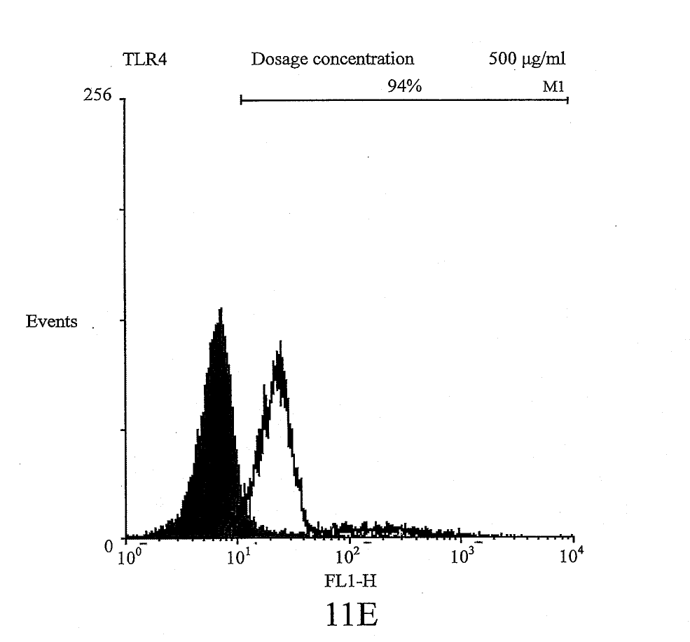

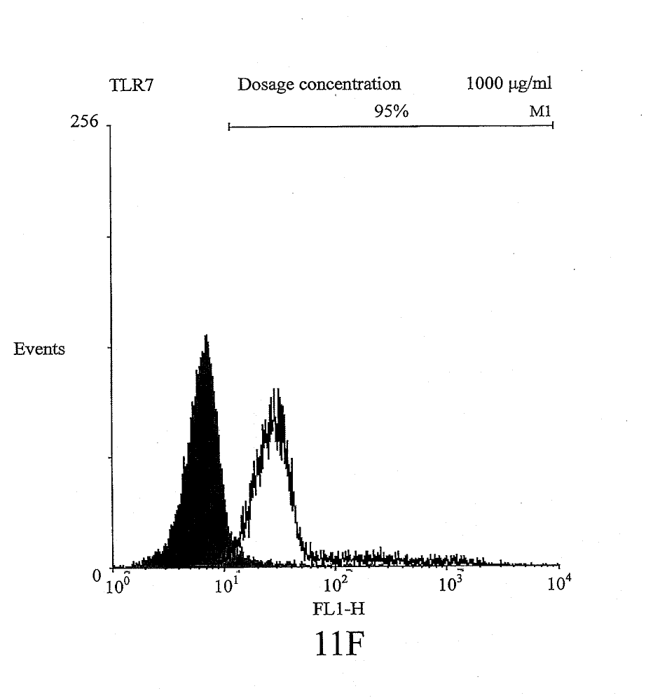

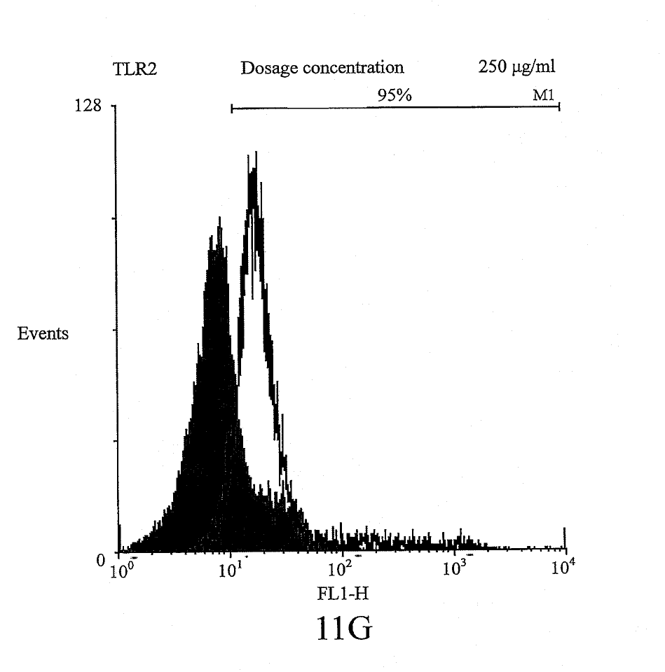

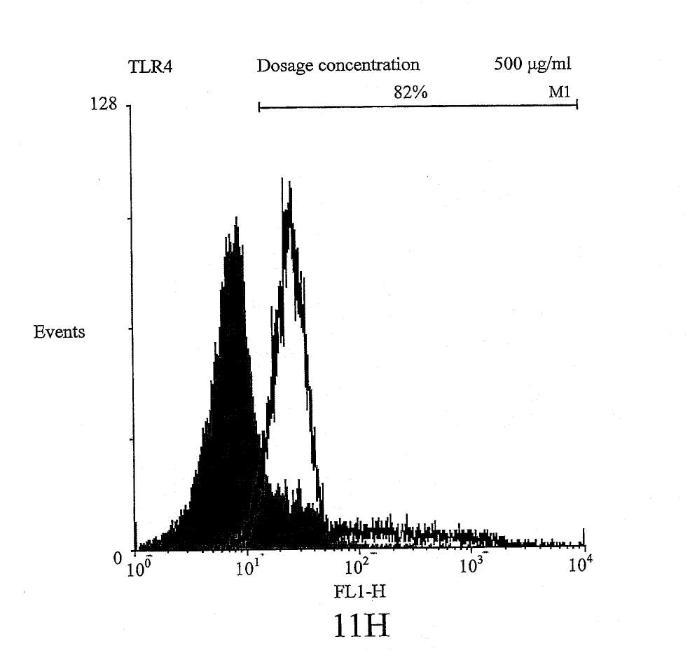

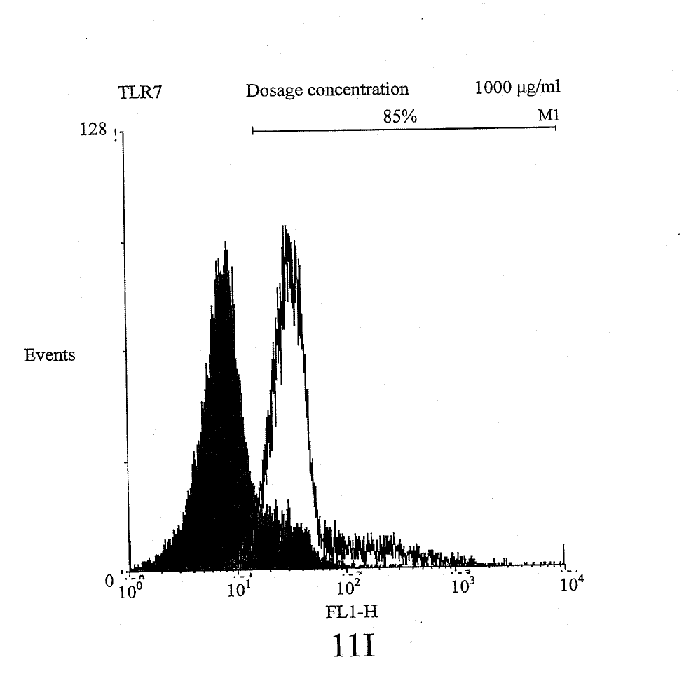

The expressions of Toll-like receptor 2 (TLR2), Toll-like receptor 4 (TLR4) and Toll-like receptor 7 (TLR7) activated by the herbal extract (prepared by Example 8) were tested. The results are shown in FIGS. 11A-11I. When a low dosage (250 μg/mL) of the herbal extract was applied, expressions of above 75% for the TLR2, TLR4 and TLR7 were achieved.

While the invention has been described by way of example and in terms of the preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An herbal extract which induces immune cells to produce interferon, extracted from an effective amount of raw material comprising Glycyrrhizae Radix, Bupleuri Radix and Scutellariae Radix, wherein Glycyrrhizae Radix, Bupleuri Radix and Scutellariae Radix have a weight ratio of 1-5:1-5:1-5.

2. The herbal extract which induces immune cells to produce interferon as claimed in claim 1, wherein Glycyrrhizae Radix, Bupleuri Radix and Scutellariae Radix have a weight ratio of 1-2:1-2:1-2.

3. The herbal extract which induces immune cells to produce interferon as claimed in claim 1, wherein the raw material further comprises an effective amount of Schisandrae Fructus and Paeoniae Rubra Radix.

4. The herbal extract which induces immune cells to produce interferon as claimed in claim 3, wherein Glycyrrhizae Radix, Bupleuri Radix, Scutellariae Radix, Schisandrae Fructus and Paeoniae Rubra Radix have a weight ratio of 1-5:1-5:1-5:1-3:1-3.

5. The herbal extract which induces immune cells to produce interferon as claimed in claim 4, wherein Glycyrrhizae Radix, Bupleuri Radix, Scutellariae Radix, Schisandrae Fructus and Paeoniae Rubra Radix have a weight ratio of 1-2:1-2:1-2:1-2:1-2.

6. A method for preparing an herbal extract which induces immune cells to produce interferon, comprising:
providing an herbal composition comprising an effective amount of Glycyrrhizae Radix, Bupleuri Radix and Scutellariae Radix, wherein Glycyrrhizae Radix, Bupleuri Radix and Scutellariae Radix have a weight ratio of 1-5:1-5:1-5;
extracting the herbal composition with a solvent to form an extract solution;
concentrating the extract solution to form a concentrated product;
drying the concentrated product; and
adding an excipient to prepare a specific formulation.

7. The method for preparing an herbal extract which induces immune cells to produce interferon as claimed in claim 6, wherein the herbal composition further comprises an effective amount of Schisandrae Fructus and Paeoniae Rubra Radix.

8. The method for preparing an herbal extract which induces immune cells to produce interferon as claimed in claim 7, wherein Glycyrrhizae Radix, Bupleuri Radix, Scutellariae Radix, Schisandrae Fructus and Paeoniae Rubra Radix have a weight ratio of 1-5:1-5:1-5:1-3:1-3.

9. The method for preparing an herbal extract which induces immune cells to produce interferon as claimed in claim 8, wherein Glycyrrhizae Radix, Bupleuri Radix, Scutellariae Radix, Schisandrae Fructus and Paeoniae Rubra Radix have a weight ratio of 1-2:1-2:1-2:1-2:1-2.

10. The method for preparing an herbal extract which induces immune cells to produce interferon as claimed in claim 6, wherein the solvent comprises water or 0.1-95% ethanol.

11. The method for preparing an herbal extract which induces immune cells to produce interferon as claimed in claim 6, wherein the solvent and the herbal composition have a weight ratio of 6:1-10:1.

12. The method for preparing an herbal extract which induces immune cells to produce interferon as claimed in claim 6, wherein the concentrated product has a solid content of 10-30%.

13. The method for preparing an herbal extract which induces immune cells to produce interferon as claimed in claim 6, wherein the concentrated product is dried by vacuum drying, freeze drying, spray drying or fluidized bed drying.

14. The method for preparing an herbal extract which induces immune cells to produce interferon as claimed in claim 6, wherein the excipient comprises starch, maltose, lactose, sucrose, mannitol, magnesium stearate, silicon dioxide, microcrystalline cellulose, carboxymethyl cellulose or talc powder.

15. The method for preparing an herbal extract which induces immune cells to produce interferon as claimed in claim 6, wherein the specific formulation comprises capsule, tablet, powder or fluid.

16. An herbal extract of activates Toll-like receptors, extracted from an effective amount of raw material comprising Glycyrrhizae Radix, Bupleuri Radix and Scutellariae Radix, wherein Glycyrrhizae Radix, Bupleuri Radix and Scutellariae Radix have a weight ratio of 1-5:1-5:1-5.

17. The herbal extract of activates Toll-like receptors as claimed in claim 16, wherein the herbal extract activates Toll-like receptor 2, Toll-like receptor 4 and Toll-like receptor 7.

18. The herbal extract of activates Toll-like receptors as claimed in claim 16, wherein Glycyrrhizae Radix, Bupleuri Radix and Scutellariae Radix have a weight ratio of 1-2:1-2:1-2.

19. The herbal extract of activates Toll-like receptors as claimed in claim 16, wherein the raw material further comprises an effective amount of Schisandrae Fructus and Paeoniae Rubra Radix.

20. The herbal extract of activates Toll-like receptors as claimed in claim 19, wherein Glycyrrhizae Radix, Bupleuri Radix, Scutellariae Radix, Schisandrae Fructus and Paeoniae Rubra Radix have a weight ratio of 1-5:1-5:1-5:1-3:1-3.

21. The herbal extract of activates Toll-like receptors as claimed in claim 20, wherein Glycyrrhizae Radix, Bupleuri Radix, Scutellariae Radix, Schisandrae Fructus and Paeoniae Rubra Radix have a weight ratio of 1-2:1-2:1-2:1-2:1-2.

22. A method for preparing an herbal extract of activates Toll-like receptors, comprising:
providing an herbal composition comprising an effective amount of Glycyrrhizae Radix, Bupleuri Radix and Scutellariae Radix, wherein Glycyrrhizae Radix, Bupleuri Radix and Scutellariae Radix have a weight ratio of 1-5:1-5:1-5;
extracting the herbal composition with a solvent to form an extract solution;
concentrating the extract solution to form a concentrated product;
drying the concentrated product; and
adding an excipient to prepare a specific formulation.

23. The method for preparing an herbal extract of activates Toll-like receptors as claimed in claim 22, wherein the herbal composition further comprises an effective amount of Schisandrae Fructus and Paeoniae Rubra Radix.

24. The method for preparing an herbal extract of activates Toll-like receptors as claimed in claim 23, wherein Glycyrrhizae Radix, Bupleuri Radix, Scutellariae Radix, Schisandrae Fructus and Paeoniae Rubra Radix have a weight ratio of 1-5:1-5:1-5:1-3:1-3.

25. The method for preparing an herbal extract of activates Toll-like receptors as claimed in claim 24, wherein Glycyrrhizae Radix, Bupleuri Radix, Scutellariae Radix, Schisandrae Fructus and Paeoniae Rubra Radix have a weight ratio of 1-2:1-2:1-2:1-2:1-2.

26. The method for preparing an herbal extract of activates Toll-like receptors as claimed in claim 22, wherein the solvent comprises water or 0.1-95% ethanol.

27. The method for preparing an herbal extract of activates Toll-like receptors as claimed in claim 22, wherein the solvent and the herbal composition have a weight ratio of 6:1-10:1.

28. The method for preparing an herbal extract of activates Toll-like receptors as claimed in claim 22, wherein the concentrated product has a solid content of 10-30%.

29. The method for preparing an herbal extract of activates Toll-like receptors as claimed in claim 22, wherein the concentrated product is dried by vacuum drying, freeze drying, spray drying or fluidized bed drying.

30. The method for preparing an herbal extract of activates Toll-like receptors as claimed in claim 22, wherein the excipient comprises starch, maltose, lactose, sucrose, mannitol, magnesium stearate, silicon dioxide, microcrystalline cellulose, carboxymethyl cellulose or talc powder.

31. The method for preparing an herbal extract of activates Toll-like receptors as claimed in claim 22, wherein the specific formulation comprises capsule, tablet, powder or fluid.
